(19) United States
(12) Patent Application Publication
(10) Pub. No.: US 2023/0324994 A1
(43) Pub. Date: Oct. 12, 2023

(54) HAPTIC DEVICE

(71) Applicant: NEWSOUTH INNOVATIONS PTY LIMITED, Sydney, New South Wales (AU)

(72) Inventors: Thanh Nho DO, Chester Hill, New South Wales (AU); Mai Thanh THAI, Chester Hill, New South Wales (AU); Phuoc Thien PHAN, Chester Hill, New South Wales (AU); Trung Thien HOANG, Chester Hill, New South Wales (AU); Nigel Hamilton LOVELL, Coogee, New South Wales (AU)

(21) Appl. No.: 18/042,393

(22) PCT Filed: Aug. 20, 2021
(86) PCT No.: PCT/AU2021/050922

§ 371 Date: Feb. 21, 2023

(30) Foreign Application Priority Data
Aug. 21, 2020 (AU) 2020902997

(51) Int. Cl. G06F 3/01

(52) U.S. Cl. CPC G06F 3/016

(57) ABSTRACT

A haptic device comprising: an array of soft hydraulic skin stretch devices, and soft microtubule muscles configured to control the skin stretch devices.

### RELATED APPLICATION

The present disclosure claims benefit of priority to Australian Provisional Patent Application Number: 2020902997 filed 21 Aug. 2020 the contents of which are incorporated herein by reference. In jurisdictions where incorporation by reference is not permitted, the applicant reserves the right to add any or the whole of the contents of said Application Number: 2020902997 as an Appendix hereto, forming part of the specification.

### FIELD OF THE INVENTION

The disclosure relates to haptic devices and specifically to novel skin stretch devices for encoding haptic places.

### BACKGROUND OF THE INVENTION

Frequently, communication of information to human beings is achieved via language that may be conveyed using visual or audial modalities. However, in many circumstances, visual and audial cues may be disruptive. Haptics—the sense of touch—offers humans the ability to explore surrounding environments and manipulate objects in daily activities. Haptic simulation provides an alternative way to convey information. Moreover, haptic simulation may assist in controlling the balance of amputees, providing motion guidance and navigational assistance, allowing surgical training, and providing tactile alphabets. In surgical procedures, surgeons could take benefits from real-time force feedback to prevent excessive force applied to tissues. In medical training, effective haptic devices can be used to enhance the interaction between medical students and clinicians or provide a useful tool to carry complex training for surgical procedures within virtual organs without accessing real human tissues.

One known method for achieving haptic motion guidance is to apply force feedback to the skin via devices that are mechanically grounded through rigid linkages. However, this method frequently requires a large workspace, along with bulky, and expensive tools.

Wearable devices, in contrast, offer compact, flexible, lightweight, and low-cost solutions. However, the mechanics of existing wearable devices interacting with human skin still pose many challenges, which severely impact their usability due to the occlusion of rigid components.

Among the many types of haptic devices, vibrotactile displays, which apply vibrations to the skin using vibration motors or linear resonant actuators, are the most widespread and investigated, because of the ease of design and actuation. Several methods to provide directional cues via vibrotactile feedback have been described in the literature. To use vibration feedbacks, users must learn an association between a localized vibration cue and a spatial direction which make them difficult to localize because cutaneous vibrations spread efficiently throughout the skin surface, leading to desensitization and sensory adaptation, which can impair device functionality over time. In addition, continuously wearing or touching vibrating devices causes the users' desensitization and discomfort after a long period of time. The effective sensation of vibration feedback can be even decreased when users are in motion.

Skin stretch devices (SSDs) which apply tangential force to the skin via a tactor provide a haptic place that may not be limited to force, motion, direction, stiffness, indentation, and surface geometry.

In some known systems, skin stretch (SS) feedback has been used to guide human motion, render friction or stiffness of the objects or surface geometry. Several groups have investigated haptic feedback based on tangential force to the skin via a tactor in the form of wearable devices. SS is a known part of the normal physiological apparatus for proprioception, contributing to the human sense of motion and location of limbs. The motions and velocities required to impart skin stretch can be slow, allowing the design of compact, low power, and portable devices. Skin stretch cues are perceived by the sensory system through slowly adapting type II (SA-II) stretch sensitive afferent nerve fibres that are widespread in tissues associated with hairy skin deformation has been shown as a promising haptic feedback modality in many studies in the last decade because of its superior features. First, SSD may impact human skin in a way which is similar to natural haptic interactions. Second, SS feedback may provide 3-D directional cues by combing omnidirectional shear force and one normal force that have been proven by several studies. Third, SSDs may be useful in guiding users without distracting visual or auditory sense.

Although several wearable haptic devices have been developed, most studies have focused on wearable tactile feedback devices for the fingertips because the glabrous skin has the great density and different types of mechanoreceptors. These haptic devices are able to deliver normal and tangential skin deformation to the finger pad by moving an end effector against the surface of the finger pad while the device itself is grounded to the finger. Despite advances, these devices could not provide effective shear forces to communicate with the skin due to the use of rigid components or low energy efficiency of actuation systems such as onboard rigid DC motors or tendon-driven mechanisms.

Moreover, most existing skin stretch devices are rigid and therefore are not able to conform to the human skin to induce effective shear forces, which prevents their use in many haptic applications.

Any discussion of the background art throughout the specification should in no way be considered as an admission that such art is widely known or forms part of common general knowledge in the field.

### SUMMARY OF THE INVENTION

It is an object of the invention, in its preferred form to provide an improved form of haptic device.

In accordance with a first aspect of the present invention there is provided an actuator device including: a central housing frame having a planar cavity; a central tactor surface located within said planar cavity; a series of controllable synthetic muscles elements attached to the housing frame and to the central tactor surface and activated to allow for controlled movement of around the planar cavity; and activation means for activating the controllable synthetic muscles so as to move the tactor around the cavity in a controlled manner.

In some embodiments, the synthetic muscles include at least one microtubular actuator which comprises a central elongated flexible conduit surrounded by a helical coil and the synthetic muscles are activated by controlling the pressure of a fluid within the conduit. The synthetic muscles can include multiple microtubular actuators intertwined or twisted together.

In some embodiments, the central housing is in the form of a ring. The central housing frame can be attached to a flexible planar sheet material. In some embodiments, the microtubular actuator further includes an outer flexible sheath. The synthetic muscle elements can include an initial pre-set elongation element for setting an initial tension in said microtubular actuator.

In some embodiments the actuator further comprises an out of axis resiliently deformable element providing out of axis movement of the tactor upon activation of the synthetic muscle elements. The element can include a balloon. The out of axis resiliently deformable element can be independently controlled to provide out of axis deformation. The number of synthetic muscle elements can be at least four, interconnecting the central tactor surface with the frame.

In some embodiments, the synthetic muscle elements are formed within a flexible sheet.

Disclosed is a haptic device comprising an array of soft hydraulic skin stretch devices, and soft microtubule muscles configured to control the skin stretch devices. In some forms the device comprises a tactor configured to deliver 3-axis tangential forces from the soft microtubule muscles to the skin. In some forms the tactor is soft and may be composed of soft silicone elastomer. In some forms the haptic device is integrated into a textile. In some forms the textile is in the form of a wearable garment.

This invention in some forms introduces novel and soft, hydraulic SSDs that can induce the 3-axis tangential forces to the skin via a tactor. In some forms the SSDs may be pneumatic. In some forms the SSDs are hand worn. The invented SSDs are controlled by new soft microtubule muscles (SMMs) which are driven by hydraulic pressure via custom miniature syringes and DC micromotors. The soft 3-axis SSDs can generate repeatable, high-speed, omnidirectional shear forces and desired displacement to induce a wide range of haptic sensations. The SMMs, SSDs created in this invention will enable new forms of haptic communication to augment human performance during daily activities such as tactile textual language, motion guidance, and navigational assistance, remote surgical systems, rehabilitation, education, training, entertainment, or virtual and augmented reality.

In some forms in use the skin stretch devices are able to deliver forces that are both normal and tangential to the skin.

Also disclosed is a soft microtubule muscle driven by a fluid pressure source. In some forms the soft microtubule muscle consists of a flexible silicone microtube or microtubule and a hollow micro-coil which is made from inextensible fibers connected with a source of fluid pressure.

### BRIEF DESCRIPTION OF THE DRAWINGS

Embodiments will now be described by way of example only, with reference to the accompanying drawings in which:

FIG. 1 shows a soft 3-axis skin stretch device of one embodiment;

FIG. 2 shows the skin stretch device of FIG. 1 without textile;

FIG. 3 to FIG. 6 shows an SSD incorporated into a fabric and worn by a finger;

FIG. 7 shows use of an SSD in a textile with balloon inflation which generates the normal force in z-axis;

FIG. 8 and FIG. 9 show a SSD device to be worn by a finger;

FIG. 10 to FIG. 13 show the SSD in use within a housing;

FIG. 14 illustrates a first from of formation of a soft microtubule muscles for use in a skin stretch device;

FIG. 15 illustrates a further form of soft microtubule muscles;

FIG. 16 illustrates form of manufacture of soft microtubule muscle;

FIG. 17 illustrates a twisted form of soft microtubule muscles;

FIG. 18 illustrates one form of testing of soft microtubule muscles;

FIG. 19 illustrates the testing of a microtubule actuator;

FIG. 20 illustrates axial force testing of a microtubule actuator;

FIG. 21 to FIG. 25 illustrates testing results for testing of a microtubule actuator;

FIG. 26 and FIG. 27 illustrate an initial pre-set elongation mechanism;

FIG. 28 is a graph of actuation results;

FIG. 29 illustrates a testing apparatus;

FIG. 30 shows a fabrication process for the outer sheath of the soft microtubule muscles;

FIG. 31 shows various positions of a tactor head in a SSD;

FIG. 32 shows an alternative embodiment of a 2 axis SSD;

FIG. 33 shows an alternative embodiment of a SSD;

FIG. 34 and FIG. 35 shows an embodiment of an SSD in which the tactor incorporates an inflatable balloon;

FIG. 36 illustrates an exploded view of an array of SSDs arranged and integrated into flexible fabric/textile layers;

FIG. 37 shows a plurality of applications of an SSD;

FIG. 38 shows an array of soft 3-axis SSDs in a steering wheel;

FIG. 39 shoes haptic motion guidance for (a) translation and rotation; (b) multitasks of multi fingers; (c) motion of wrist; (d) motion of arm.

FIG. 40 and FIG. 41 shows a plurality of applications including medical and specifically remote medical applications;

FIG. 42 and FIG. 43 shows textual patterns induced by SSD;

FIG. 44 shows an array shear-force display;

FIG. 45 shows potential application areas for soft skin stretch devices;

FIG. 46 and FIG. 47 shows a skin stretch device driven by four soft microtubule muscles and its integration into the flexible fabric layers

FIG. 48 to FIG. 50 show a further embodiment of a soft tactor integrated into a 3-axis SSD in flexible fabrics and on the human index finger;

FIG. 51 and FIG. 52 show further embodiments of a housing for a SSD including a tactor.

### DETAILED DESCRIPTION

Disclosed in some forms are haptic devices comprising soft microtubular muscles and hydraulic skin stretch devices that may in some forms be worn on the body and in particular on the hand. In some forms of the present disclosure, the haptic devices may allow normal and shear forces to be delivered to skin areas in ways that are similar to what is felt in the real world.

In some forms, the haptic devices allow control of force threshold and motion which may be beneficial as the way the users sense shear force highly relies on their mechanoreceptor density and the skin stretch device distribution over the skin surface. In some forms the device may be in the form of a finger worn soft haptic device that may effectively induce skin deformation.

In some forms, the invented skin stretch devices are integrated into flexible fabrics to enhance the interaction between the device and human skin, similar to human clothing. In addition, the disclosed skin stretch devices may simultaneously induce 2-D shear force in x-y plane driven by soft microtubular muscles and a normal z-axis force to the contact skin via an adjustable tactor.

In some forms, the SSDs are controlled by new soft microtubule muscles (SMMs) which are driven by hydraulic pressure via custom miniature syringes and DC micromotors. The soft 3-axis SSDs can generate repeatable, high-speed, omnidirectional shear forces and desired displacement to induce a wide range of haptic sensations. First, the invented soft muscles and 3-axis skin stretch devices can conform to the skin geometry, which is essential to assist with routine activities or provide uninterrupted tactile communication during movement. Second, they are able to match with the geometric complexity of the skin's surface without occlusion by rigid components. Therefore, the encoding of information by multiple SSDs at a desired area of the skin is possible. Third, current SSDs are not able to produce both normal and tangential forces to the skin, which is required to increase the degrees of freedom (DOFs) and bandwidth. Our invented technologies can overcome these limitations. Fourth, our technologies are scalable and can be manufactured as large arrays while maintaining bandwidth and force output. Fifth, our devices can be integrated into flexible fabric/textile to form arrays or soft haptic skin interfaces (SHSIs) with distributed tactile stimulation. Therefore, our invention will enable new forms of haptic communication to augment human performance during daily activities such as tactile textual language, motion guidance, and navigational assistance, remote surgical systems, rehabilitation, education, training, entertainment, or virtual and augmented reality.

In some forms, disclosed is a new type of muscle that can transmit force and motion from a far distance without scarifying its input energy. The developed SMM presented in this work is driven by fluid pressure source that consists of a flexible silicone microtube or microtubule and a hollow micro-coil which is made from inextensible fibers. The obtained muscle composite is lengthened or shortened under the applied fluid pressure. Fluid transmission tube or guide tube which connects the inner channel of the soft microtubule to a miniature syringe and a micro DC motor (Faulhaber, Germany) via a linear ball screw system (MISUMI, Japan) is used to supply the pressure. By constraining the radial expansion of the flexible microtubule via micro-coil, it is possible to mechanically program the muscle to perform desired motions and forces.

In some forms, to enhance the interaction between the device and the wearers, SSDs will be integrated into a flexible fabric layer that allows the devices to be worn like human clothing. The sliding tactor is made from soft silicone elastomer (composite between Sylgrad 184, Down Corning and Ecoflex 0030, Smooth-On Inc.) to enhance the interaction force between the SSDs and the user's skin. The tactor which is connected to the four SMMs can slide over the skin surface to induce the skin stretch sensation. The relative position of tactor with respect to the fabric housing is controlled by miniature syringes remotely located far away from the working areas via the four SMMs which are arranged in a cross shape where the tactor bottom head is used as a 4-way connector. At the beginning of the assembly, each SMM is elongated to reach a level of 50% of its maximum strain so that a balancing force is maintained. To control the motion in the desired direction, individual SMM is controlled in pressurized, depressurized or initial states, depending on the movement of its associated syringe plunger. By controlling the pressures applied to the muscles, we are able to control the tactor position within the working space rw as well as normal force to the skin by adjustable tactor head.

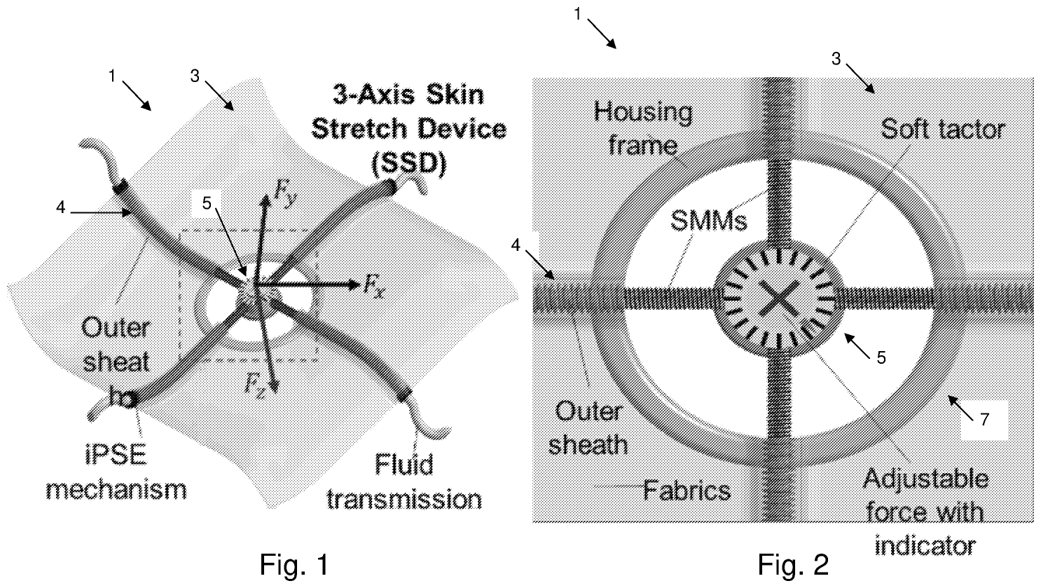

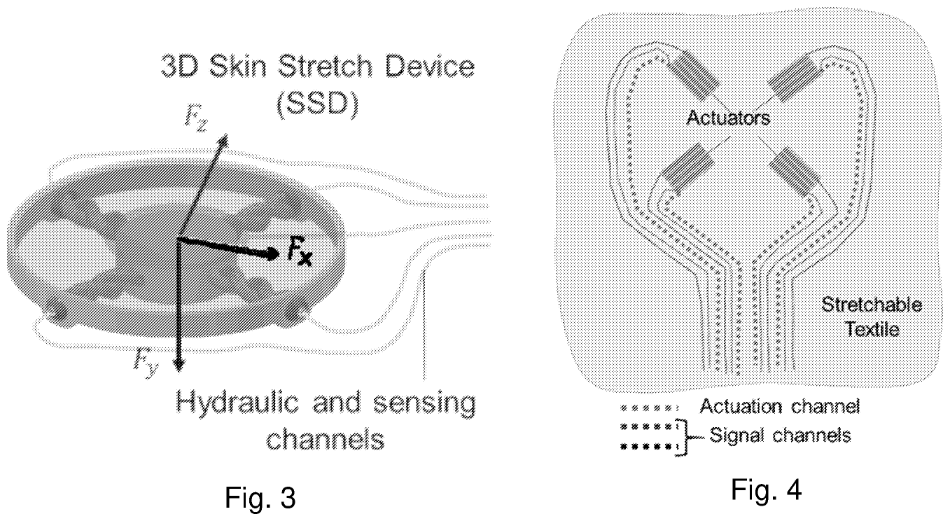

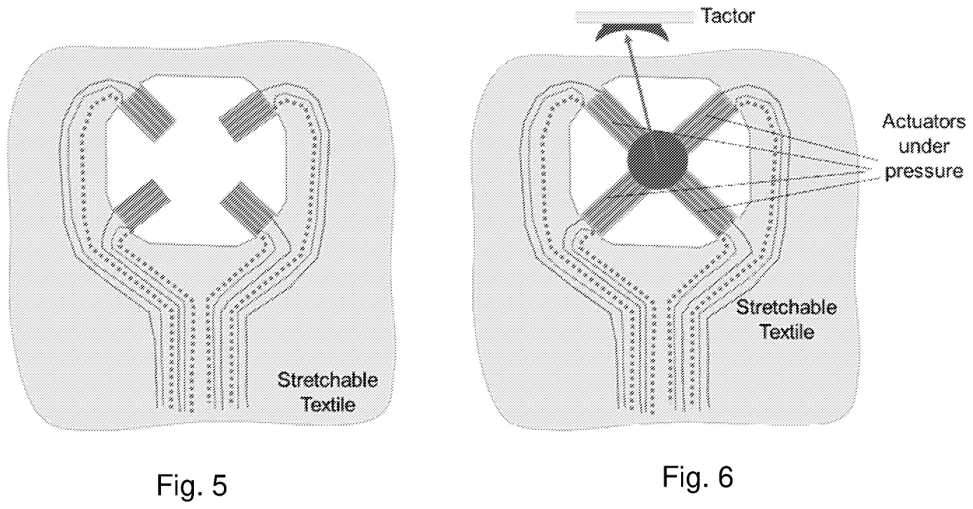

Referring now to FIG. 1 and FIG. 2, disclosed in some forms is a soft 3-axis skin stretch device 1 where the shear forces (Fx, Fy) to the skin are controlled by soft microtubule muscles via a tactor.

In the embodiment shown in FIGS. 1 and 2, the skin stretch device 1 is integrated into a flexible fabric 3. The skin stretch device includes a plurality of soft microtubule muscles 4 which are engaged with a soft tactor 5. In some forms, the skin stretch device comprises four soft microtubule muscles extending outwardly from the soft tactor 5. In some forms the soft microtubule muscles are spaced apart around the soft tactor. To deliver accurate skin stretch sensation to the skin via a tactor, a normal force Fz in the z-axis driven by a soft adjustable tactor 5 is used.

In some forms the skin stretch device 1 further comprises a housing frame 7. In some forms the soft microtubule muscles 4 extend from the tactor 5 to the housing frame 7

The sliding tactor 5 in some forms is made from soft silicone elastomer (for example a composite of Sylgrad 184, Dow Corning and Ecoflex 0030, Smooth-On Inc.) The soft silicon tactor may enhance the interaction force between the skin stretch device and the user's skin. The tactor 5 is configured such that it is connected to the four soft microtubule muscles 4 and can slide over the skin surface to induce the skin stretch sensation. The relative position of the tactor 5 with respect to the fabric housing is controlled by a series of miniature syringes remotely located far from the working areas via the four soft microtubule muscles. In the illustrated form the soft microtubule muscles are arranged in a cross shape where the tactor bottom head is used as a 4-way connector.

In some forms, during assembly, each soft microtubule muscles is elongated to reach a level of 50% of its maximum strain so that a balancing force is maintained. To control the motion in the desired direction, an individual soft microtubule muscle is controlled in pressurized, depressurized or initial states, depending on the movement of its associated syringe plunger. By controlling the pressure applied to the muscles, the tactor position within the working space rw as well as normal force to the skin is monitored by an adjustable tactor head.

For example, at the center pressures P1=P2=P3=P4=P0 and contraction forces generated by SMM are balanced \vec{F_{P1}}=\vec{F_{P2}}=\vec{F_{P3}}=\vec{F_{P4}}, while at the position H, the applied pressures to the SMMs P1<P2, P3, P4 with their contraction forces \vec{F_{P1}}=\vec{F_{P2}}+\vec{F_{P3}}+\vec{F_{P4}}. At position I, P1, P4<P2, P3, and therefore the SMM forces are \vec{F_{P1}}+\vec{F_{P4}}=\vec{F_{P2}}+\vec{F_{P3}}. Note that P0 is the balanced pressure applied to the SMMs when the tactor is located in the center of the working space with radius rw and P1, P2, P3, P4 are the pressures insides the SMM 1, 2, 3, 4, respectively. F_{P1}, F_{P2}, F_{P3}, F_{P4} are the contraction forces produced by SMM 1, 2, 3, 4, respectively.

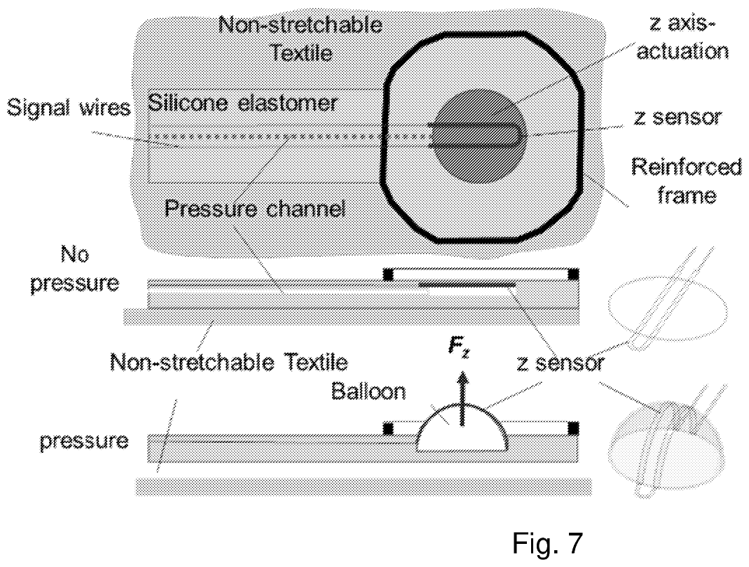

As shown in FIG. 7 also disclosed is a working principle of the soft tactor with balloon inflation as the z-axis actuator and embedded liquid metal channel as the sensing element.

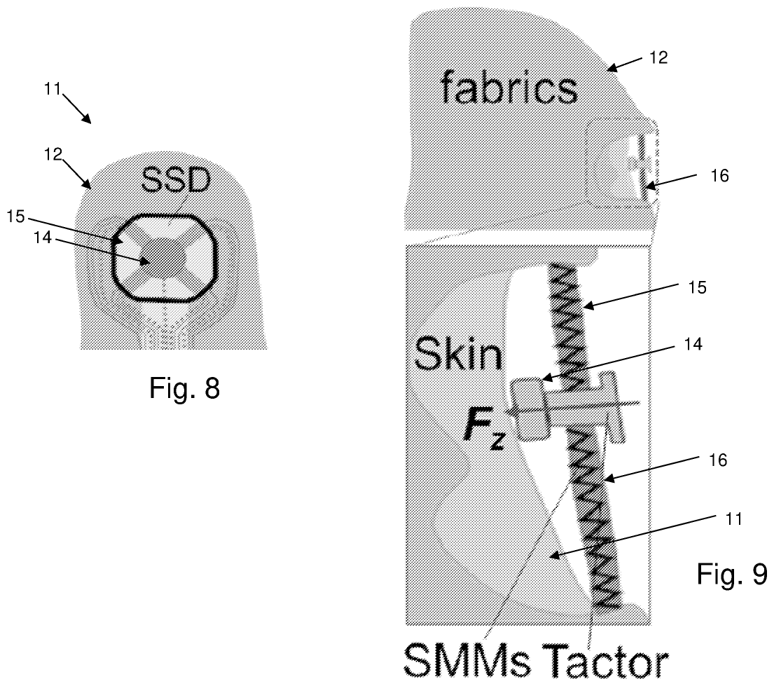

Referring to FIG. 8, disclosed is a skin stretch device 11 incorporated into a wearable fabric article 12. The skin stretch device comprises a tactor 14 which is positioned in an aperture 15 in the fabric 12 such that the tactor can be in contact with the skin of the user. In other forms the tactor may be in indirect contact with the skin of the user.

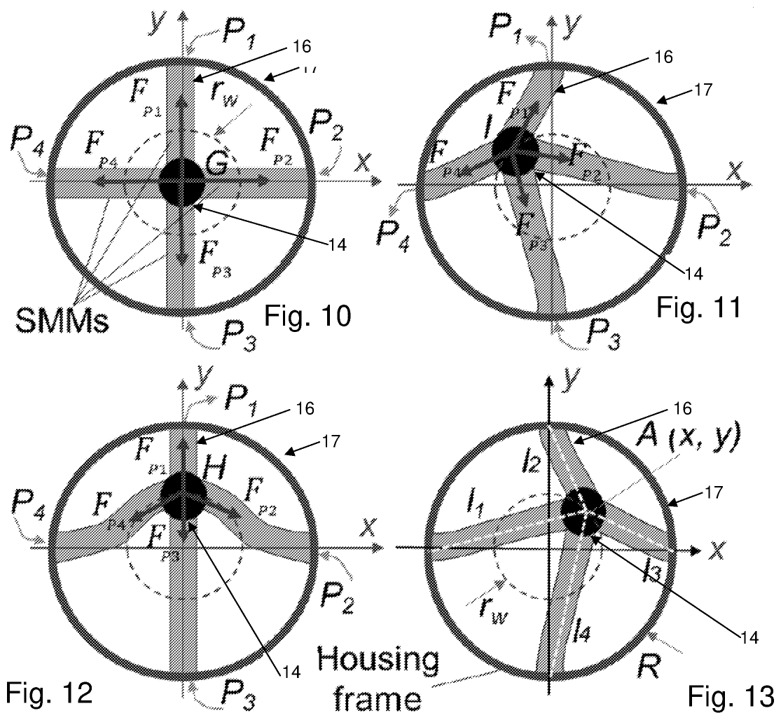

Referring to FIG. 10 through FIG. 14, the skin stretch device comprises a tactor 14 which is connected with a plurality of soft microtubule muscles 16 which are positioned in an array about the tactor and spaced apart around the tactor. The tactor and microtubule muscles are positioned in a housing 17 which supports the device and allows the muscles to stretch in multiple directions under force providing contact and feedback with the skin. The figures show the following: FIG. 10 shows a tactor head at the balanced state; FIGS. 11 and 12 show motion of the tactor controlled by the four soft microtubule muscles and FIG. 13 shows calculation of the soft microtubule muscle length with respect to tactor location.

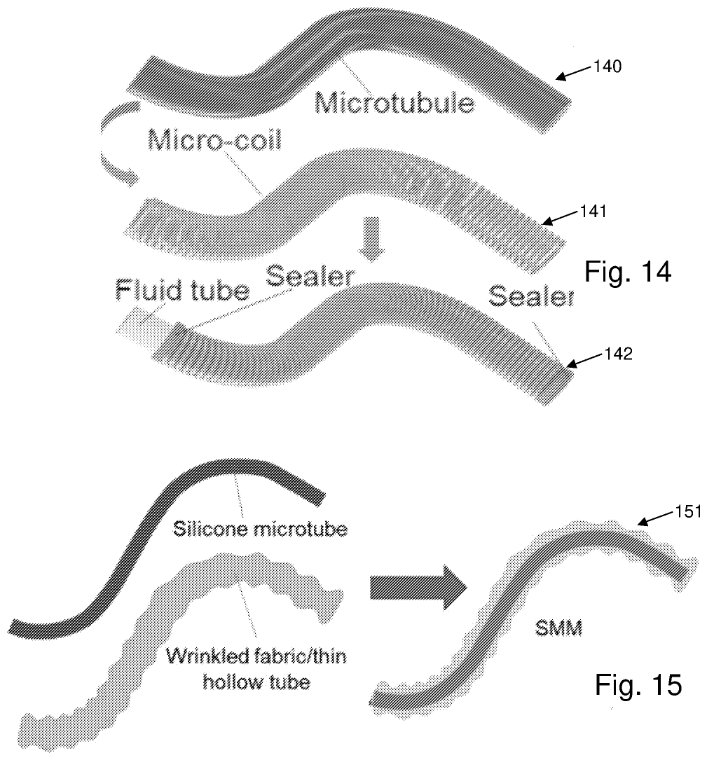

Referring now to FIG. 14, there is illustrated the formation process for forming a microtubule. The arrangement is formed around a soft core 140 around which is wound a coil 141. The resulting soft microtubule muscle 142 is then sealed. Referring to FIG. 15, the soft microtubule muscle may have a wrinkled configuration among other configurations and embodiments. In some forms the wrinkled configuration of wrinkled fabric about an internal stretchable silicon microtubule may offer stronger shear force for haptic applications at a shorter length while maintaining compact size without using outer sheath and initial pre-set elongation (iPSE) mechanism. As illustrated in FIG. 15, the microtube can then be covered in a wrinkled fabric or the like to form a final soft microtubule muscle 151 for use in a skin stretch device.

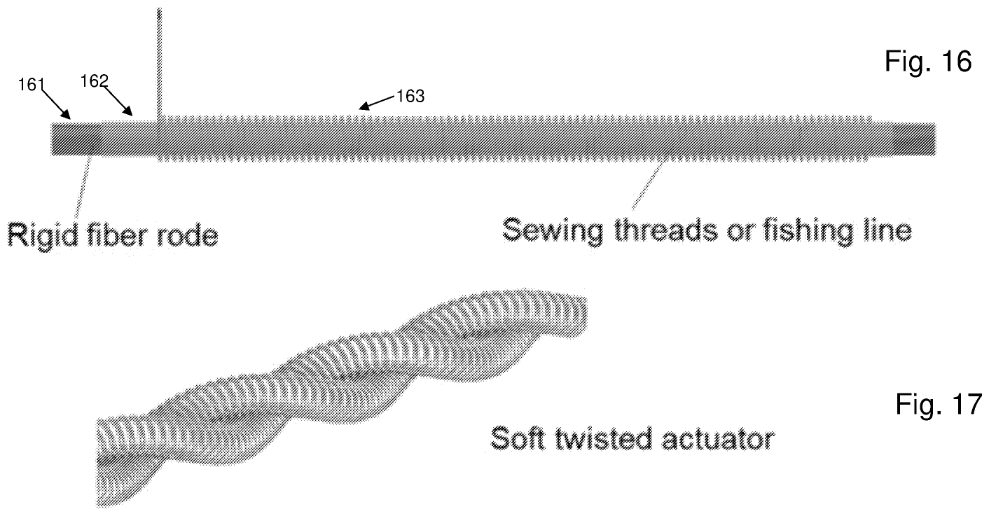

In one arrangement in FIG. 16, the microtube muscle can be formed by utilising a carbon fiber inner rod 161 to hold the silicon tube 162, around which a sewing thread or fishing line is formed.

In another embodiment as shown in FIG. 17, the soft muscles are fabricated from a twisted soft microtubule muscle.

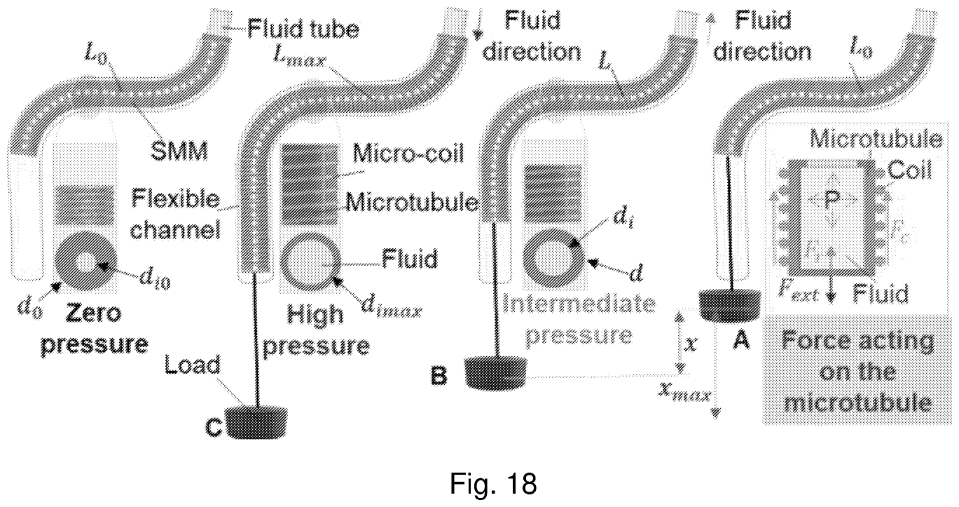

As shown in FIG. 18, the soft microtubule muscle comprises a microtubule such as a silicon tube or tubule to which a pressure is hydraulically applied. The microtubule comprises a flexible channel and in the illustrated form a microcoil extends around the diameter of the microtubule. Fluid acts on the microtubule to apply fluid pressure to the soft microtubule muscle. Sealers are located within the microtubule.

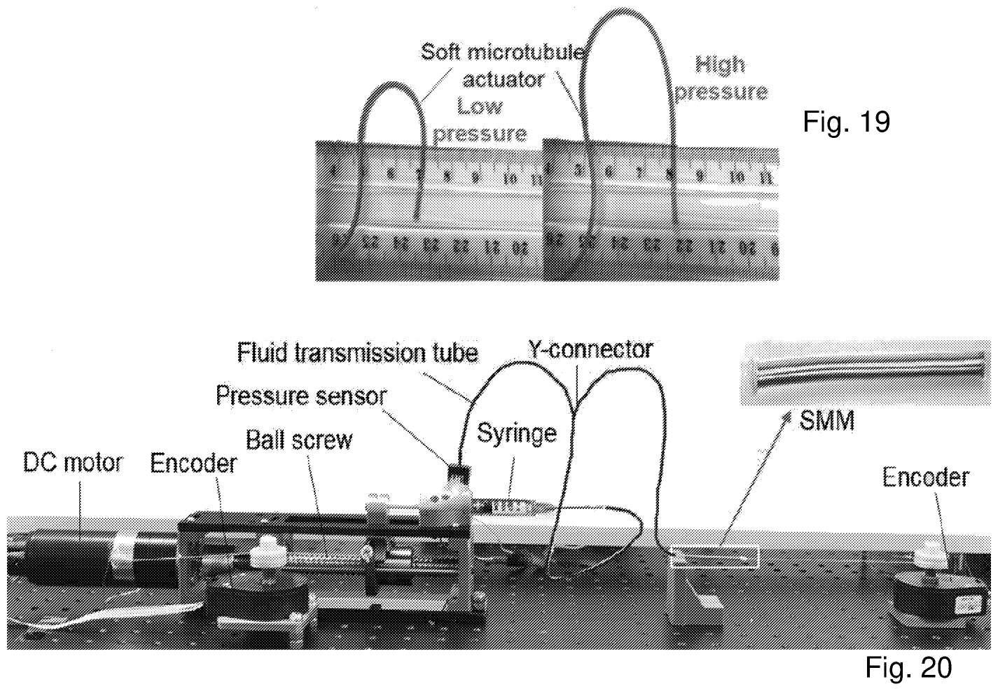

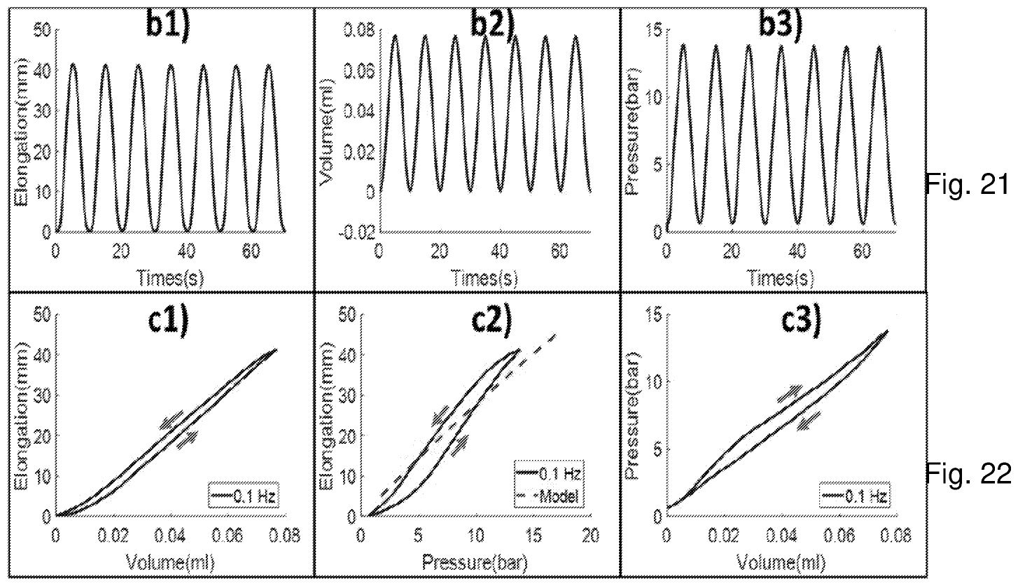

FIG. 19 shows the soft microtubule muscle at low applied pressure (left) and high applied pressure (right) respectively.

In some forms, the skin stretch device may be obtained by distributing the soft microtubule muscle in 4-configuration, following by the integration soft inflatable balloon to control the z-axis force for the skin stretch device (as shown earlier in FIG. 1). In this embodiment, conductive liquid metal (EGaIn, Galinstan) can be used to provide the force feedback from the inflatable balloon. The inflatable balloon can be made from soft silicone elastomer (Ecoflex, Dragon skin, Smooth-On or any stretchable rubber) and then the obtained balloon is integrated into the surface of non-stretchable fabric/textile.

One of the main advantages of soft microtubule muscle is the ability to control the generated force or displacement threshold while maintaining the muscle length at a fixed value via the use of initial pre-set elongation (iPSE). To create an efficient and comfortable wearable skin stretch device, the movement area of the tactor head may be maximized while its size is minimized Some factors such as the contraction force from the soft microtubule muscles and the elongation of their associated opposite soft microtubule muscles may affect the tactor motion.

### EXAMPLES

To characterize the effect of iPSE on the tactor displacement, 1-axis skin stretch devices which consist of a tactor and two soft microtubule muscles is fabricated. Each experiment is carried out with different 1-axis skin stretch device at different iPSE value for each soft microtubule muscle but a fixed nominal length for the device or (L₀₁+L₀₃) is maintained. The working range of each SDD is defined by the total length of the two soft microtubule muscles (L₀₁+L₀₃) which are connected via a tactor. As an illustration, the displacement of the tactor given at high iPSE is greater than that at a low iPSE. For each experiment, there is a 5% increment of the elongation for the SMM. To measure the tactor head position, a laser displacement sensor (Model IL-100, Keyence, USA) which is connected to a controller (QPIDe, Quanser, Canada) are used. The optimal iPSE value for the current design is 150% that will be used as an input value for the developed 3-axis SSDs. Beyond this value, the tactor displacement is decreased which can be explained by the limitation on the elastic strength of the silicone microtube and the outer micro-coil elongation. To implement the optimal iPSE into skin stretch devices in practice, each soft microtubule muscle is elongated up to 50% of its optimal iPSE value. This point is defined as an initial starting position for the tactor or balanced position.

FIG. 20 shows an apparatus for elongation testing. One end of the microtubule actuator was fixed on a 3D printed base while the other end is connected to a constant load of a force 0.1N via an optical encoder (S6S-1000-IB, US Digital, USA) to record the position of the SMM distal end. The working fluid was supplied via BD Luer-Lok™ 1-mL syringe (inner diameter of approximate 4.5 mm) driven by a DC micromotor (model 3272G024CR with encoder 1E3-1024, Faulhaber, Germany) and a ball screw mechanism (MISUMI, Japan). To mitigate the nonlinear hysteresis from the DC gearhead and ball screw system, another optical encoder (S6S-1000-IB, US Digital, USA) is utilized to record the syringe plunger displacement. Fluid pressure is monitored using a pressure sensor (40PC Series Sensor, Honeywell, USA). To eliminate the pressure loss during the operation, the pressure sensor is located as close as to the muscle port via a Y-connector. The soft muscle is driven by using sinusoidal fluid displacement at a frequency of 0.1 Hz (slow motion for skin stretch applications) while fluid pressure ranging from 1 bar to 15 bar. The signals are decoded synchronously using a data acquisition device (QPIDe Data Acquisition Device, Quanser, Canada) and MATLAB Simulink (Mathworks, Inc., USA). The signal from the pressure sensor is passed through a digital lowpass filter to eliminate high-frequency noise. The instantaneous displacement of the fluid volume is calculated from the displacement of the syringe plunger and its geometry.

FIG. 21 illustrates results of elongation—time measurements and FIG. 22 illustrates elongation volume and pressure measurements. The results indicated consistent performance over repeated actuation and close agreement with the analytical model.

The results given in FIG. 21 and FIG. 22 show that the muscle elongation increases with additional fluid volume or applied fluid pressure by moving the syringe plunger forward and vice versa for the reverse motion. This response is consistent with the elongation and fluid pressure under repeated actuation. It is noted that the relation between volume and displacement is almost linear with a small hysteresis loop while the volume-pressure and pressure-elongation relationships exhibit greater hysteresis. A notable divergence from the model is the nonlinear hysteresis due to the deformation of nonlinearly elastic materials and friction loss that are not given completely in the proposed models given by the equations below. The developed analytical model correctly predicted the observed ranges of muscle elongation and the data which means that the muscle performance is within the expected range that is demonstrated by a closer trend of the model to follow the real experimental data and even in the higher frequency of fluid movement. In addition, the result with a small hysteresis loop from the relation between applied input volume or displacement motion of the syringe plunger is good for the feedforward or feedback control when the displacement or volume is used as control input compared to that of pressure with higher hysteresis loop.

Axial Force Test

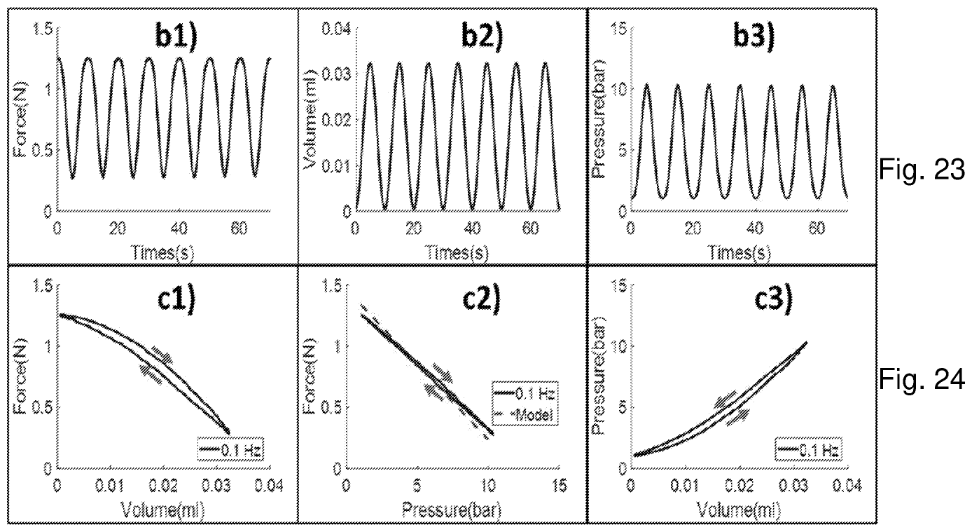

Next an axial force test was conducted. The force performance of the SMM in the axial direction using an experimental setup similar to FIG. 20. The results for these test are illustrated in FIG. 23 and FIG. 24. For this testing, a miniature FUTEK load cell 1 lb (FUTEK, USA) is used. One end of the SMM is fixed on a 3D printed base while the other end is connected to the load cell head which is mounted onto a 3D printed platform. The applied input from the miniature syringe to the muscle is similar to that of elongation testings. It is shown that the SMM performs consistency over repeated testing cycles. Particularly, the force-pressure curve exhibits a linear relation with a little nonlinear hysteresis loop while the force-volume and pressure-volume introduce a higher hysteresis loop. The analytical models given by the equations below reveal a qualitatively good agreement with the experimental data. This means that in the force mode control, the pressure can be used as control input instead of volume or displacement. In addition, the pressure information can be used to provide a good prediction of the output force under the absence of an onboard force sensor. This plays a vital role in the development of SMM-based compact wearable haptic devices and other applications where the use of bulky components to provide the output feedback is avoided. Although the hysteresis loops for the force-volume and pressure-volume curves are well recorded, there is a strong nonlinearity in both forward and backward directions. It is noted that this developed SMM provides a stronger force compared to other soft pneumatic and hydraulic actuator with the same size because the generated force from SMM comprises of two components: (i) the storage elastic energy (STE) from the inner silicone tube and (ii) the restoring force from the micro-coil while existing soft actuators only rely on STE.

Experiments were carried out to examine the maximum elongation of the SMMs so that they can well suit to be implemented into the developed SSDs and other wearable haptic and assistive systems. The size and geometry of testing SMM are similar to that of elongation and force test. To determine the maximum elongation level, the fluid is pressurized from a miniature syringe to the SMM until it fails. While the soft microtube has high elongation at break, the maximum elongation of SMM highly depends on the outer micro-coil (size of fibers and the coil pitch). Experimental results show that the flexible silicone microtube has a high elongation at break of 750% (Ecoflex 0030) while the constrained micro-coil only elongate up to 200%. However, most failures come from the sealer and connector at both ends of the SMM which is due to the weak interface between the rigid component (fluid transmission tube) and soft material (silicone microtube). To ensure that the failure does not originate from these mechanical factors, a stronger adhesive sealer at the connectors is highly desired. SMM strain is anticipated to reach the elongation limit of micro-coil until ballooning occurs in between the two ends of the SMM which is due to the silicone microtube reaches its elastic material limit under high applied stress and strain in the sense that the connectors and sealers are sufficiently strong to maintain the contact. However, the developed SSDs only require approximately 1 mm to move the tactor and induce desired skin stretch sensation and therefore an elongation of approximately 100% is sufficient. To determine the soft actuator length used in the skin stretch device, experiments with trials and errors are carried out before the fabrication of the SSD.

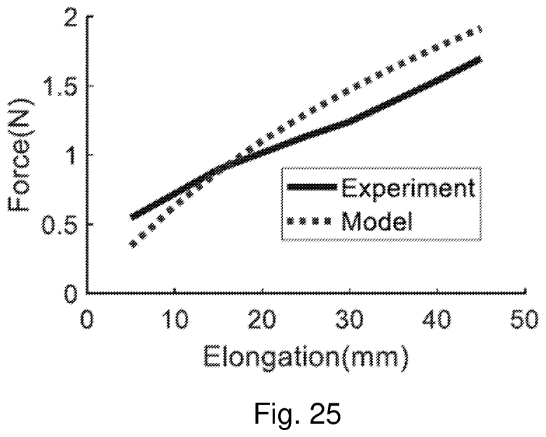

The relationship between the elongation and the corresponding maximum force in real experiments is investigated and validated. Then the obtained measured data and the developed model are compared. The comparison result is given in FIG. 25. Here, an initial length of around 45 mm plus 20 mm for each soft muscle is selected to be used in the SSD as it can generate a sufficient tangential force (around 1N as shown in the literature [27) in order to induce a shear force sensation to the human skin at the finger. The result reveals that the actual data is close to the developed model, but the maximum force obtained from the developed model is slightly higher that of measured data. This can be attributed to the loss caused by sliding friction and the dissipative energy in the experiment.

Experiments are carried out to evaluate the SMM durability overtimes in order to ensure that the performance of the SMM-based SSD is stable during its operation. A sinusoidal signal is applied for each test (elongation and force) until the SMM reaches a maximum elongation of 40 mm and a maximum force of 1.2 N, respectively. The performance of the soft actuator was consistent over the testing period although there is an approximate 0.4% reduction in the elongation and a 5% reduction in the axial force. This reduction can be attributed to the stress relaxation of the inner elastic tube, so this device can provide a roughly similar skin-stretch effect on fingertip skin after many cycles.

As the skin stretch velocity is one of the main factors for mechanoreceptors discharges rates [28]. Therefore, the maximum speed of tactor head movement at maximum iPSE value for SMMs (˜150% elongation) is evaluated. The speeds are recorded using a Keyence displacement sensor. The performance of 10 trials is recorded with the highest velocity applied to the DC motors-driven miniature syringes by withdrawing the fluid in one SMM while adding the fluid into the other SMM. Results show that the average speed is approximately 12.30 mm/s while the maximum speed is around 20.14 mm/s.

It is noted that the soft microtubule muscles used in real-time experiments may buckle if the lengthened ones exceed a threshold beyond their balanced position. To better describe this phenomenon, the balanced force and applied pressure to the tactor are analyzed, F_{P1}=F_{P3} and pressures P1=P3=P0. Experiments show that if the applied pressure to SMM1 or SMM3 is increased to a value that is higher than a threshold (P1 or P3>P0), unexpected buckling occurs in the depressurized muscle (SMM1 or SMM3). As the soft microtubule muscles are controlled by independent syringes while the skin stretch device is working in open-loop mode, it is challenging to control the opposite soft microtubule muscles to the desired position. In order to avoid unexpected buckling, a pressure threshold is applied for each actuated soft microtubule muscles so that the DC motors can be controlled within this threshold.

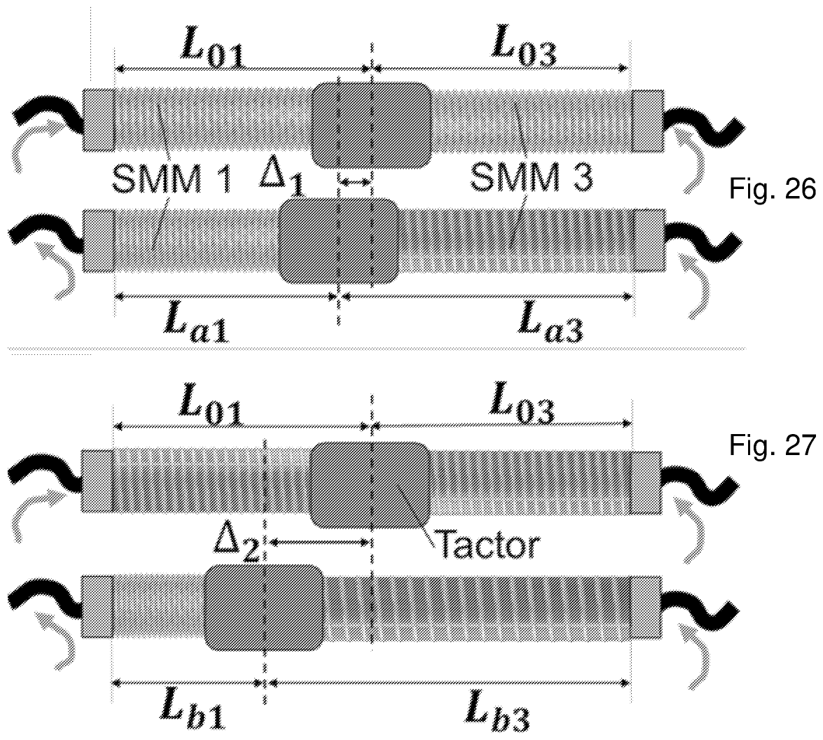

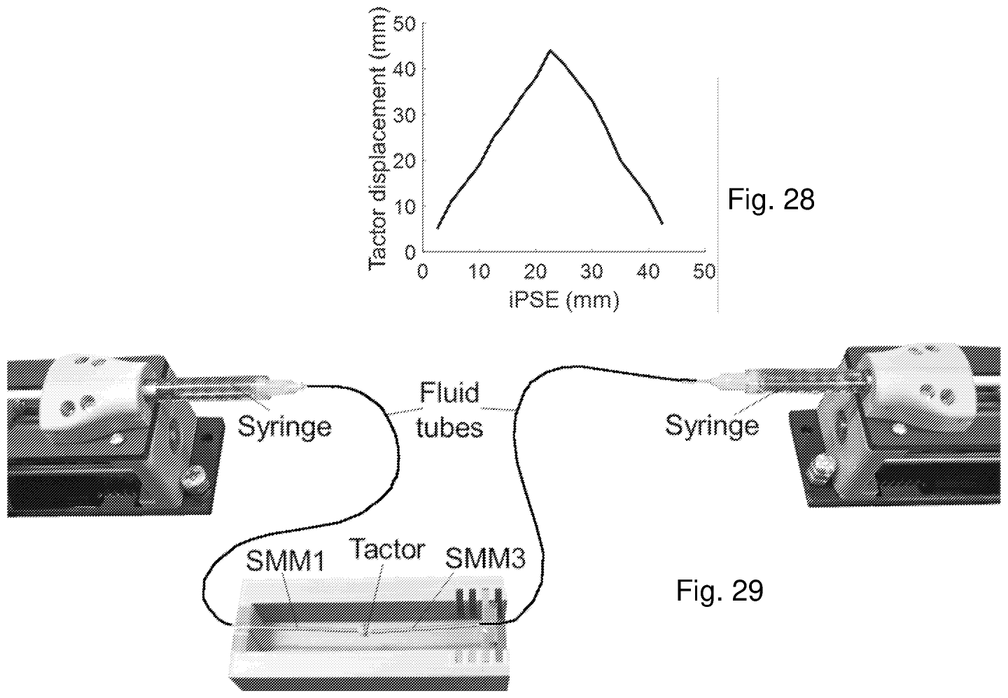

Referring to FIG. 26 and FIG. 27, in some forms to control the force threshold to the soft microtubular muscles, an initial pre-set elongation mechanism is introduced. FIG. 26 and FIG. 27 show different tactor displacements of the 1-axis SSD with different initial elongation of the SMMs. FIG. 28 shows the experimental results for the maximum displacement of the tactor head attached in two soft actuators for various initial pre-set elongation.

FIG. 29 illustrates the test device for undertaking the measurements.

### Example Fabrication of Outer Sheath and iPSE Mechanism

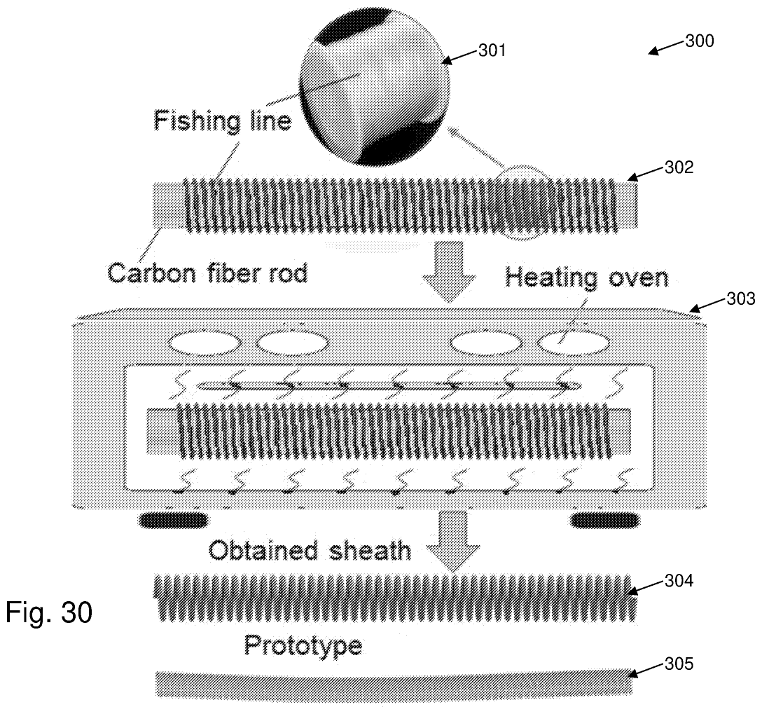

Referring to FIG. 30, there is disclosed one method of fabricating an outer sheath for a soft microtubule muscle. The method involves fishing line 301 which is wrapped around a carbon fiber rod 302 and heated 303 to obtain a sheath 304.

To guide the soft microtubule muscles within the fabric layers, outer sheath mechanisms may be used. The sheath mechanism is a type of hollow plastic coil springs that are made from the 0.5 mm fishing line (Jarvis Walker, Australia). To fabricate the outer sheath, the fishing line is wrapped around a carbon fiber rod as shown in the figure (1.43 mm in outer diameter, CST, Composite Store Inc., CA, USA). To maintain the helical shape, the composite is placed into an oven 303 at 1300 C for 2 hours. Next, use a hollow carbon fiber rod (inner diameter of 1.5 mm, CST, Composite Store Inc., CA, USA) to release the coil. Only uniform coils are chosen to use in the wearable haptic device. To adjust the iPSE value for the SMMs, an iPSE mechanism that consists of an actuator holder and spacers is developed. As the fishing coil and soft microtubule muscles are highly flexible, they can be easily integrated into the fabric layers so that users do not have any discomfort with rigid components.

As shown in the illustrated embodiments, the soft microtubule muscle-driven 3-axis skin stretch device may in some forms consist of a plurality of soft microtubule muscles, a soft tactor, a wearable fabric housing such as a stretchable textile, initial pre-set elongation (iPSE) mechanisms, and outer sheaths. In some forms the plurality of soft microtubule muscles comprises 4 microtubule muscles. To enhance the interaction with the users, in some forms the skin stretch devices may be integrated into a flexible fabric layer that allows the devices to be worn like human clothing.

Kinematic Model of the Skin Stretch Device Tactor

To characterize the skin stretch device tactor, a proposed kinematic model represents the relationship between the position of tactor A (x, y) and the working length of each soft microtubule muscle l₁, l₂, l₃, l₄ within the housing frame R. The tactor head only reaches the radius of r_{w}<R due to the constraint of this frame, the size of soft microtubule muscles and the tactor radius. The working length of each soft microtubule muscles (SMM) within a radius R is expressed by:

$$\{ \begin{matrix}{l}_{1} = \sqrt{{y}^{2} + {( x + R )}^{2}} \\ {l}_{2} = \sqrt{{x}^{2} + {( R - y )}^{2}} \\ {l}_{3} = \sqrt{{y}^{2} + {( R - x )}^{2}} \\ {l}_{4} = \sqrt{{x}^{2} + {( y + R )}^{2}}\end{matrix} \{ \begin{matrix}{l}_{1} = \sqrt{Δ + 2 Rx} \\ {l}_{2} = \sqrt{Δ - 2 Ry} \\ {l}_{3} = \sqrt{Δ - 2 Rx} \\ {l}_{4} = \sqrt{Δ + 2 Ry}\end{matrix} , \qquad ( 1 )$$

where Δ=R²+x²+y²

The actual length of each SMM (Lₘᵢ), i=1, 2, 3, 4 which depends on the tactor position at A (x, y) is given by:

$$\{ \begin{matrix}{L}_{m 1} = {L}_{0} - ( R - {l}_{1} ) \\ {L}_{m 2} = {L}_{0} - ( R - {l}_{2} ) \\ {L}_{m 3} = {L}_{0} - ( R - {l}_{3} ) \\ {L}_{m 4} = {L}_{0} - ( R - {l}_{4} )\end{matrix} , \qquad ( 2 )$$

where L₀ is the initial length of each SMM.

This kinematic equation can be applied for both SMMs and twisted configuration for the muscles in SSDs.

One design in some forms of the skin stretch device is that each SMM slides inside a hollow coil as a sheath embedded into flexible fabrics. This enables the SMM to be controlled by a remote external hydraulic source via long, narrow, and tortuous paths, eliminating bulky on-board actuator and allowing the compact design of a simpler and smaller haptic device for the fingertip. Although this configuration is inspired by the tendon-sheath mechanism, the proposed approach offers better performances in terms of energy efficiency due to the smaller loss of nonlinear friction and hysteresis. The design does not limit the motion of its wearer because the fabric housing is highly conformal and adaptable to the skin surface. In addition, the developed device does not prevent the relative movement of the two consecutive knuckles.

E. SMMs-Driven 2-Axis Skin Stretch Device

In some forms, skin stretch feedback is capable of conveying position and spatial direction via a small tactor, which in some forms may be just a few millimeters in diameter. When lifting an object, the perception of the weight of the vessel is primarily mediated by cutaneous afferents that sense the strain of the skin. The motions and velocities required to impart skin stretch can be slow, allowing the design of compact, low power, and portable devices. To meet the displacement requirement for cutaneous haptic feedback, the capability of the SSD tactor for directional and positional cues are investigated. In some forms, four SMMs-driven 2-axis SSDs with an optimal iPSE value of 100% for the muscles is fabricated. In some forms, four DC micromotors (model 3272G024CR with encoder 1E3-1024, Faulhaber, Germany) may be connected to ball screw mechanisms (MISUMI, Japan) and used to control the SMMs via BD Luer-Lok™ 1-mL syringes as the preferred embodiment. Fluid pressure is monitored using a pressure sensor (40PC Series Sensor, Honeywell, USA). Experiments are conducted with a maximum displacement of the 2-axis SSD tactor within its working area via the evaluation of its reachability for eight different directions.

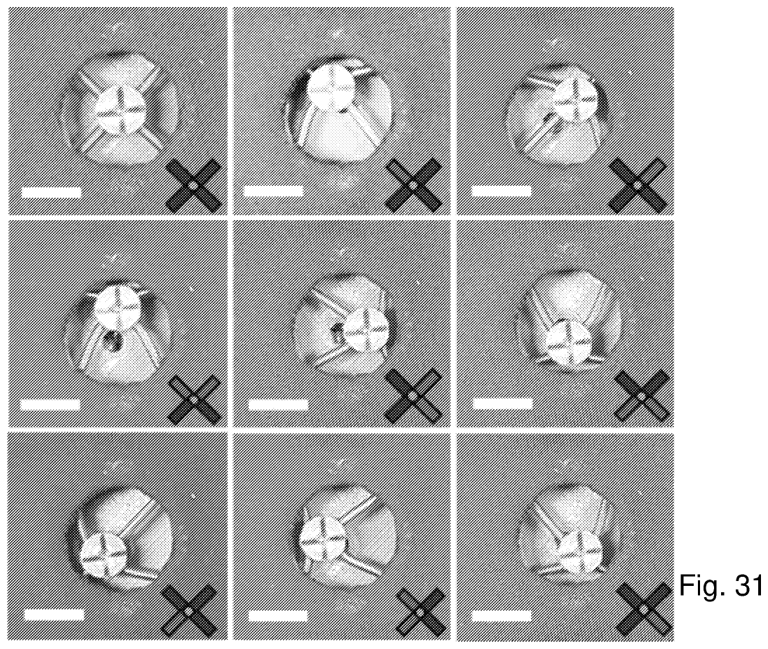

As shown in FIG. 31, the tactor head is able to reach a target position in desired directions. It is noted that the tactor can reach a circle of 9 mm within the working area of 10 mm in diameter. This demonstrates that the SSD tactor can achieve 90% of the theoretical working area.

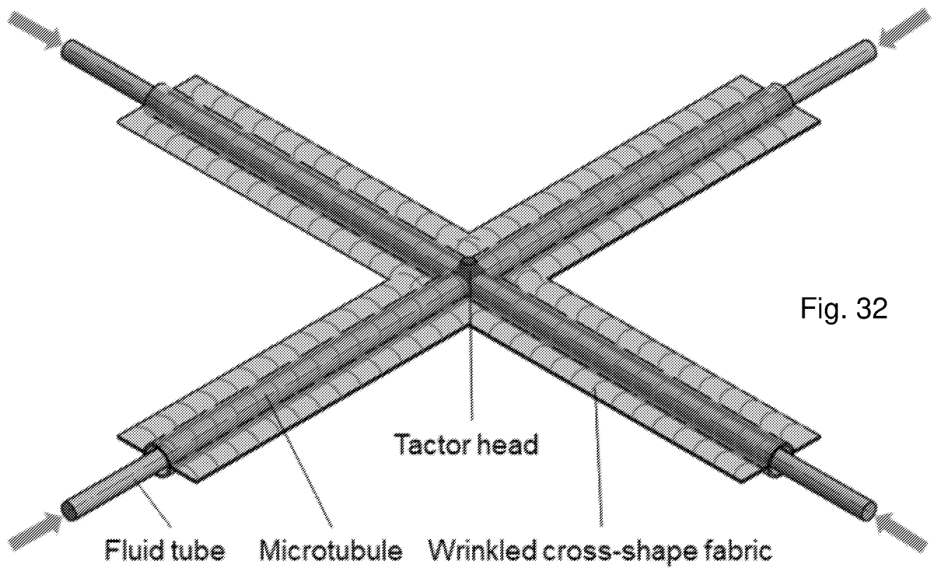

There are several different configurations, which are different from the embodiment shown above, to fabricate SMMs-driven 2-axis SSD. In an embodiment, these soft muscles are separately constructed by inextensible wrinkled fabric which is used as the constrained outer layer and microtubule as presented in the design and fabrication of soft microtubule muscle part. The composites are sewed into one piece of fabric as 4-ways connecter and then the tactor is attached to the top of the fabric as shown in FIG. 32. In a similar embodiment, four soft microtubules are inserted into a cross-shape fabric and are connected by sealer at one end of the microtubules. The fabric layers are wrinkled, sealed, and connected to the silicone microtubules to make 4 four soft muscles.

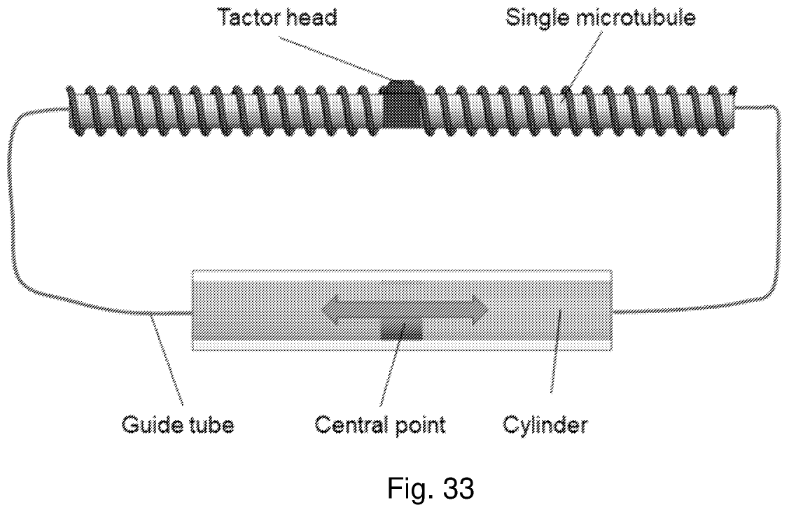

As illustrated in FIG. 33, in another embodiment, two soft muscles are controlled by a customized mechanical cylinder in the 2-axis SSD. These cylinders have a single plunger operated in bidirectional configuration of the two opposite fluid chambers with a central point connected a ball screw mechanism. The position of the tactor head depends on the location of the central points of plunger. This embodiment simplifies the design and control theory to move the tactor head in various directions using a single driving mechanism.

Shear Force Measurement

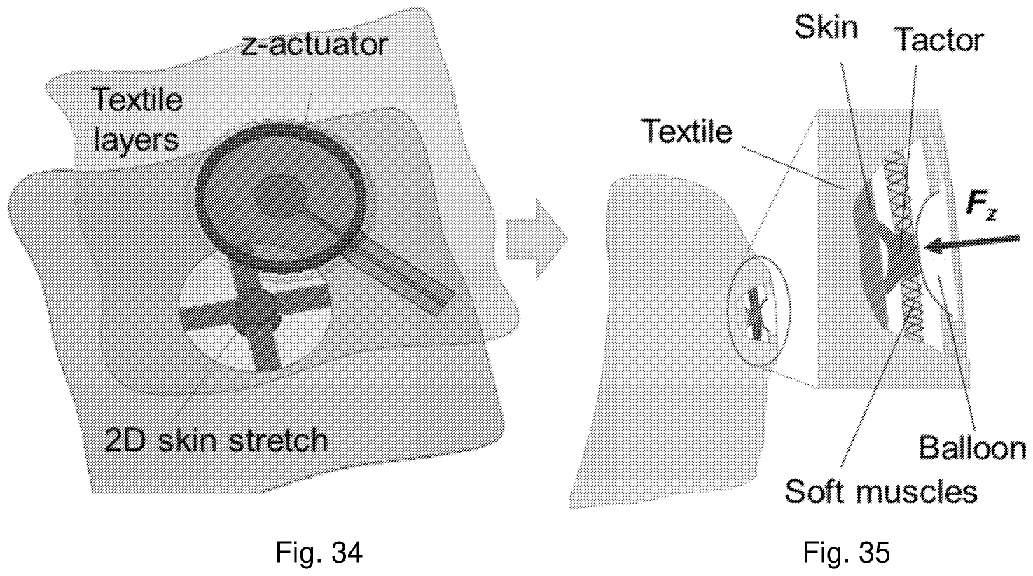

The magnitude of the shear force induced by the soft tactor in eight different directions is measured in the preferred embodiment. In this experiment, 100% for the iPSE value. A FUTEK loadcell 1 lb (FUTEK, USA) is used to measure the shear force. The maximum shear forces in eight directions are shown in FIG. 34. The results show that the shear force along diagonals is larger than that of principal directions. This can be explained by the fact that two adjacent muscles generate a higher force compared to single once. The shear forces in the opposing directions are likely different because of intrinsic inconsistencies in the fabrication process by hand. This unexpected factor can be alleviated by using the iPSE mechanism with adjustable spacers in order to make ensure the shear forces in the major axes are roughly similar at the beginning.

3-Axis SSD with Soft Tactor

In some forms of the SSD, the tactor plays a vital part to connect the four SMMs and to induce stretch sensation to the skin. To deliver accurate haptic stimuli to the skin, it is essential to control the mechanical contact state which is especially challenging for current SSDs. Adhesion of the tactor with the skin will thus be achieved via a soft silicone surface. In order to control the normal force Fz in the z-axis, an adjustable mechanism is used. The soft tactor consists of a soft silicone layer at the bottom connected to the tactor head via a 3D printed bolt. The adjustable tactor can control the strength of contact friction forces, enabling a wide range of haptic stimuli. With this new design, the SSD may be able to impart a directional shear force that can displace, deform, and stretch the skin, depending on the application such as textual communication, directional cues for motion guidance, and 3D force feedback display. To connect one end of the four SMMs to the tactor base, an adhesive glue (LOCTITE®, USA) is utilized while the other end is connected to a commercial fluid microtube (Cole-Parmer, USA). The 3D printed base is designed in a way that aligns the four force vectors from the SMM in the x-y plane. The soft tactor head which is made of ReoFlex 30 urethane rubber (Smooth-On, Inc., USA) provides high friction to improve the effect of skin stretch. The adjustable mechanism is a 3D printed bolt that can travel along the z-axis via a threaded hole in the tactor base. To achieve the desired contact force, users just simply adjust the bolt via its head. This mechanism also possesses an indicator that has a linear relation with the contact force via an adjustable angle of the bolt.

In another embodiment of the 3-axis SSD, the soft tactor can be an inflatable balloon that can provide a controllable normal force on the user's finger pad under the pressurized liquid as shown in FIG. 34 and FIG. 35. The liquid is transmitted from miniature PTFE Teflon tube that is non-stretchable and high-resistant pressure materials. The balloon is fabricated from the PET sheet or any stretchable silicone elastomer using a heat sealer or soft lithography. The inflated and deflated states can be regulated by a miniature syringe via miniature fluid transmission tube. The fluid source can be air or any incompressible ones such as water. In another embodiment, a soft conductive sensor fabricated from liquid metal alloy may be embedded in the balloon surface to sense the normal force.

The developed soft 3-axis SSD and soft tactor are also integrated into the user's finger via a hand-worn device with fabric layers. First, sewing threads were used to adhere to the SSD into fabric layers. To obtain a wearable finger device, another fabric layer was patterned in the form of a finger-like shape using a laser cutting machine and then inserted the obtained SSD composite into this fabric finger shape before reversing inside out to have the final device. The skin-stretch effect of tactor in each direction was also tested on the fingertip. Experimental results show that the tactor can move more than 1 mm when it is in contact with skin and the user does not feel any uncomfortable when wearing it for 30 minutes. The initial results demonstrate that the soft 3-axis SSD can be used to induce SS sensation to the fingers without distracting other human movements.

In an embodiment to replace the fabric housing as mentioned above for some specific applications, the housing can be soft silicone (Ecoflex or Reoflex). In this embodiment, a mould is designed and fabricated by rapid prototyping methods. Once the mould is obtained, soft muscles and the outer sheath mechanisms are inserted into the mould, following by pouring liquid silicone to create the soft housing. This invention provides several advantages because soft silicone is compliant and conformable to human finger shape and it is possible to mass production. In another embodiment, the housing can be a rigid frame that is produced by the 3D-printing method. For this embodiment, rigid frame consists of individual parts that are connected by adhesive glue. There is an adjustable mechanism using spring coils and joints to adjust the frame size so that the device can fit into various finger sizes. In another embodiment, 3D printing methods can be used to fabricate the soft housing.

Soft Haptic Skin Interfaces (SHSIs)

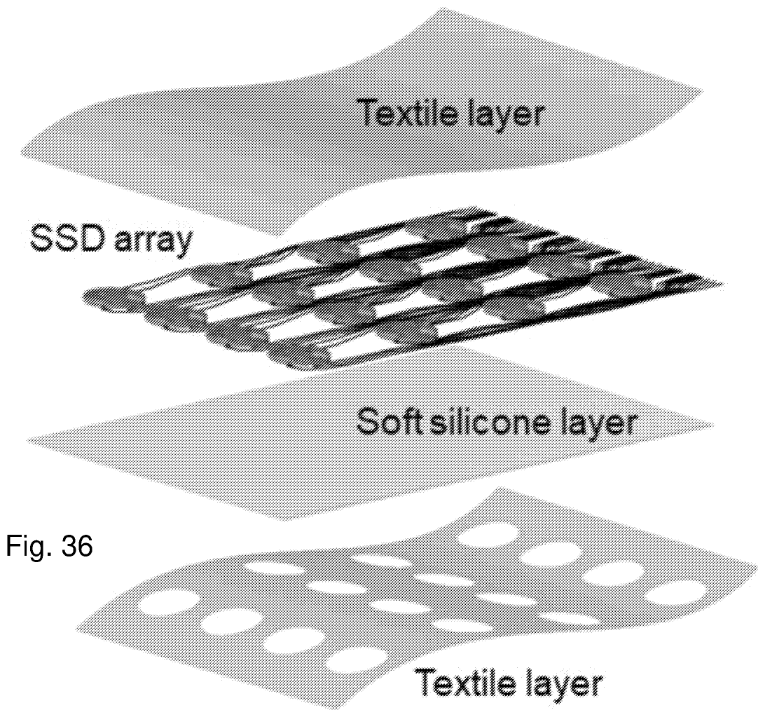

In an embodiment shown in FIG. 36, the SSDs can be arranged and integrated into flexible fabric/textile layers to form a soft haptic skin interface (SHSIs). This embodiment incorporates a large array of independent SSDs with controlled forces in 3D to effectively interact with the skin surfaces in a simple, non-invasive, reversible, and highly adaptable manner. The SHSIs are a conformal sheet that is soft and thin and it can be integrated at any desired location of the skin, which is difficult to do with current technologies. To form the array, SSDs are arranged and integrated into a predetermined position within the fabric layers using a sewing machine. In another embodiment, SHSIs are fabricated by combining twisted muscles. This embodiment offers a simpler fabrication process for the SHSIs without using outer guided sheaths and iPSE mechanisms.

Different embodiments of the housing may comprise a) a soft silicon housing, b) a 3D-printed rigid frame housing.

Applications for the Inventions

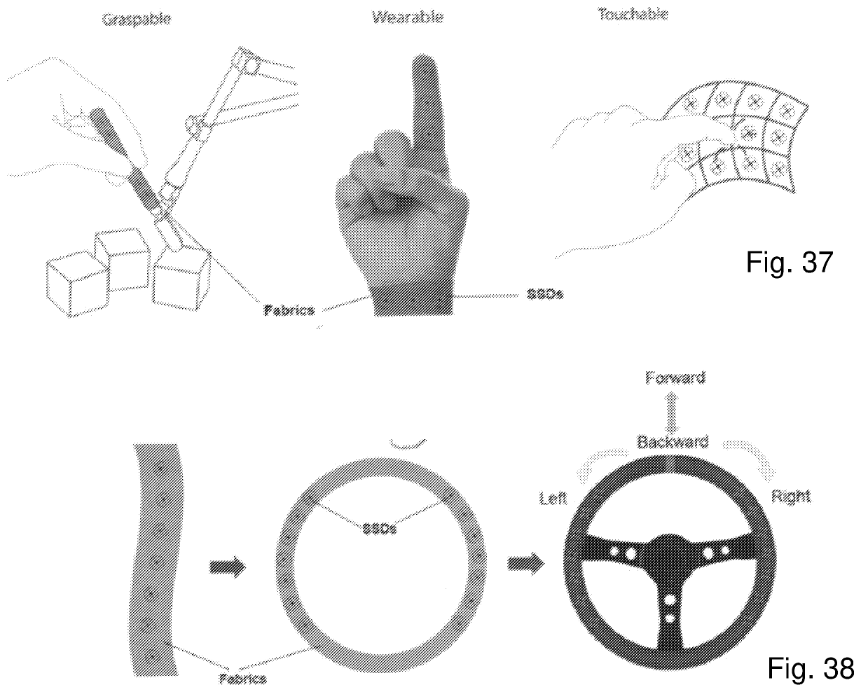

Referring now to FIG. 37, disclosed are multifunctional applications in touchable, graspable, and wearable devices. One of the advantages of the inventions is that the invented soft haptic skin interface can be implemented into various haptic applications which include graspable, wearable and touchable haptic configurations. These features may be superior when compared to present haptic devices. In one embodiment, the invented haptic interface can be wrapped around a robotic arm to form joy-stick haptic device (graspable haptic device) with skin stretch feedback. This invention can be used in surgical system, medical training, and games. In an embodiment for the graspable devices, SSDs will be distributed onto fabrics and then wrapped around grounded tools or devices that are held in the hand. In wearable devices, the invented soft haptic skin interface can be patterned in a hand-like shape or a band which can be worn by the hands or at other parts of the body to induce a cutaneous effect for displaying skin stretch sensations to the skin. For touchable application, the soft skin stretch interface can be used as a touched pad which allow the user to feel the skin stretch sensation generated from the soft skin surface, similar to touched screen in smart-phone or tablet.

A. Graspable Haptic Applications

In one example, multiple SSDs in fabrics are attached to a steering wheel on a driving car to guide the driver during the training as presented in FIG. 38. Directional information will be conveyed to the users by the integrated skin stretch devices. Each driving action will correspond to the desired pattern generated by the array of SSDs that include turning left, turning right, going forward, going backward, accelerating, and de-accelerating. In another embodiment, soft haptic skin interface can create alerts for obstacle avoidances, uneven road surface, and vibration during driving. For teleoperation in robotic surgery, the invented skin-stretch devices and their associated skin stretch interfaces can be implemented into master console in surgical and medical systems to provide cutaneous haptic feedback to the surgeons.

B. Wearable Haptic Applications

i. Haptic Motion Guidance

Skin stretch at multiple locations via tactors can produce complex haptic motion guidance by multiple directional forces and torque cues. In an embodiment of single SSDs, this device can be used as a communication mode between patients and doctors. In physiotherapy, a therapist regularly asks a patient to do something like “lift your foot a little higher” with a knee ligament repair, a common in sports. However, verbal communication is not efficient in some circumstances as the therapist has to adjust the patient's foot by hand. With the aid of skin stretch device for motion guidance, patients can be guided to move their limbs with respect to desired motions, even via remote guidance. In another embodiment, the SSDs can be combined with a mobile app and video processing software to detect human gait. In this application, people can stay at home and do relevant exercises via video guidance available in the app with support from the skin stretch haptic device.

Another application for haptic motion guidance is to support a beginner to learn piano without any instructors. The guidance device, which looks like a glove with one SSD in each figure, is able to load the songs and then it can guide the finger motion of the user to key into the keyboard. This method is named as passive haptic learning. In another application, SSD can be used for navigational assistance for the visually impaired or older people. The SSDs can help the blind navigate with complex motion because mechanoreceptor in blind people is better than a normal human

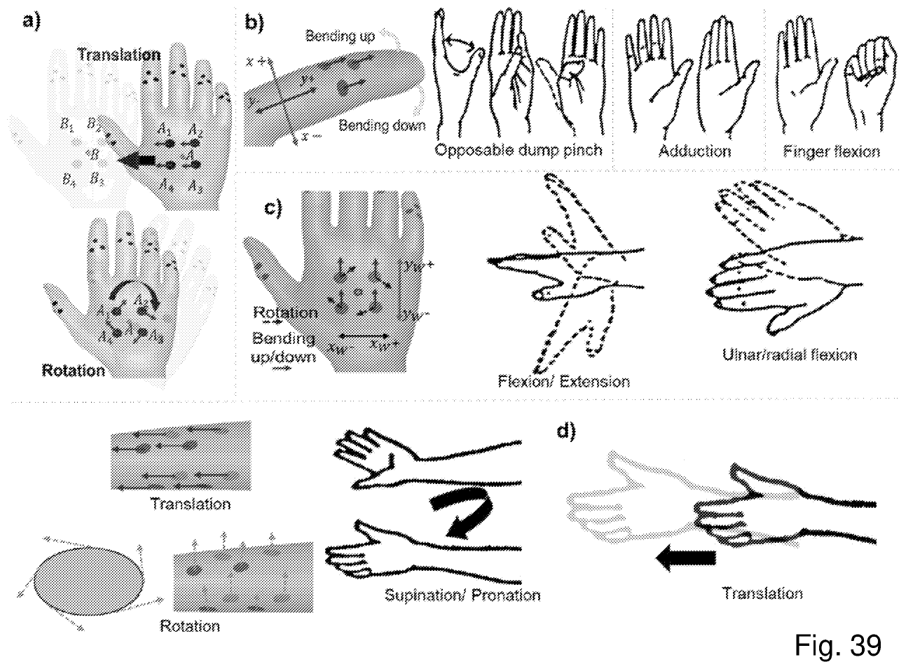

In an embodiment of SHSIs, it simultaneously induces SS cues at multiple locations to create complex haptic motion guidance such as translation (in 8 different directions), rotation cues (clockwise and counter-clockwise), and bending directions as shown in FIG. 39. This device can be placed at fingertips, forearms, wrist, and anywhere in the human body.

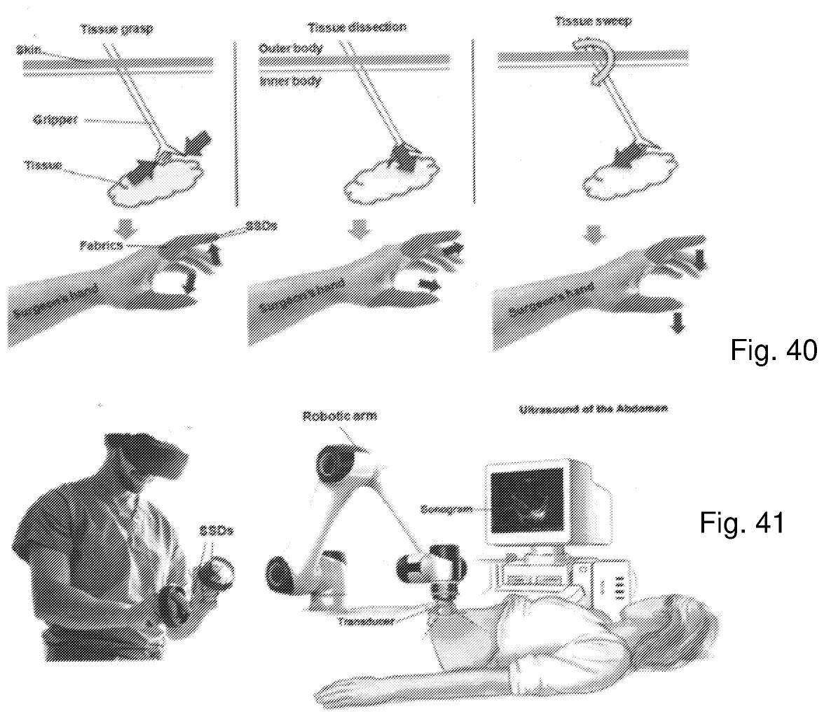

Multiple SSDs can be integrated at the fingertips to guide manual tasks including reaching, grasping, and manipulating objects in 3D, it may be useful for VR applications and medical training as shown in FIG. 40 and FIG. 41. The system may also be applied to video game controllers to augment players feeling when interacting with 3D objects like presenting their weight, surface, and shape. In education, the use of SSDs can also be particularly beneficial in any situation where the student needs to experience the simulation of realistic contact forces like force, weight from the magnetic force, gravitational force. In medical training, SS haptic devices can be used for computer interface where new doctors are trained on how to differentiate between soft tissue and hard tissue.

ii. Method for Haptic Motion Guidance

In an embodiment shown best in FIG. 39, the arrays of 3-axis SSDs are integrated into a wearable glove or any part of the human skin. The SSD arrays will be used to guide individual digits of the hand and/or human limbs in single and multi-digit movement. In this embodiment, the invented haptic guidance method will induce the haptic perception of three different directional cues including translation, rotation, and a combination of both. By independently controlling N SSDs at different locations Aᵢ, 1≤i≤N of the skin, translation, and rotation cues can be achieved. Let virtual attraction force at each tactor be \vec{f}ᵢ=kᵢ\vec{pₗ} rendered to the skin where \vec{pₗ}[m] is the position vector from tactor Aᵢ to its target Bᵢ and kᵢ is the force guidance gain, the total forces applied to the skin at the centre A from all tactors are \vec{F_{g}}=Σᵢ₌₂^{N}\vec{f}ᵢ. The skin displacement at tactor i is \vec{Δ_{ft}}=β_{fi}\vec{f}ᵢ where β_{fi} [mm/N] are the ratio of tactor motion to the guiding force. To induce the rotational cues to the skin, a virtual torque \vec{τ_{g}}=Σᵢ₌₂^{N}(Aᵢ−A)\vec{f}ᵢ about A is introduced. Through this motion guidance method, the invented SSD array is able to enable conveying arbitrary directional cues to any part of the body.

In another embodiment seen best in FIG. 39 of multi-finger motion guidance, at least 2 SSDs are distributed on the back of each finger of user's dominant hand. Instead of using torque cues induced by rotational motions from SSDs, the bending motion of each finger in this embodiment with respect to its knuckle joint is guided by forward and backward forces induced by SSDs (if forces from SSDs applied to direction y_{II}+ or y_{II}−, the finger will bend down or up, respectively Or if all forces are applied to direction x_{II}+ or x_{II}−, finger will bend left or right, respectively). By combining this motion method for fingers individually or as a group, different hand gestures can be obtained. In an embodiment, the SSD array can be distributed along the human hand to perform supination/pronation/flexion/extension and/or ulnar/radial flexion of the whole hand to prepare for grasping 3D object. This invention can be also used to guide a single finger in 8 directional cues (motion in forward, backward, bend up, bend down, twist left, twist right, tilt left, and tilt right).

iii. Tactile Alphabets and Languages

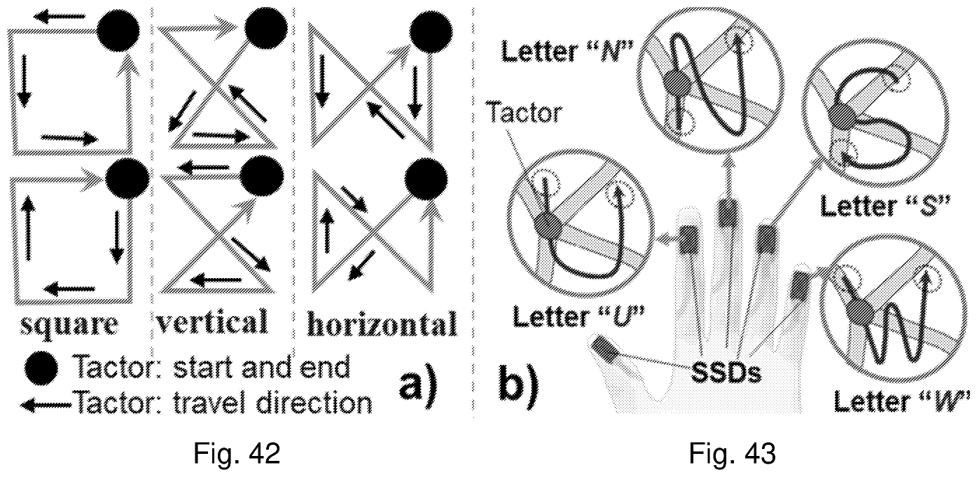

While each SSDs can generate various shapes, the combination of SSDs at multi-finger can produce words and even sentences as shown in FIG. 42 and FIG. 43. In an embodiment, several 3-axis SSDs can be integrated into a soft wearable glove. The soft glove with 3-axis SSDs integrated at the fingertips to convey tactile alphabets for visually impaired people and other means of textual communication where vision or audio is inappropriate. Tactile alphabets such as braille are hard to reproduce by a single device as users rely on the location of the dots to actively explore the letters and it requires a long learning time and bulky sheets of pattern. This embodiments can use SSDs to convey textual communication. By controlling the SSD tactor displacement with respect to the skin from multiple fingers, we are able to “draw” any letter onto the fingers or “convey” a word and then a sentence. The invented glove can provide the recognition rate of different patterns such as squares, vertical hourglasses, and horizontal hourglasses. This invented glove can convey tactile alphabets for the visually impaired when their vision and ear are unavailable, which alleviates the effort of acquiring knowledge and learning languages and to replace the complex learning method via Braille and Morse code.

iv. 3D Force Feedback Displays

In an embodiment, the invented 3-axis SSDs can be integrated into a wearable fabric glove to reproduce and amplify both normal and shear forces for several emerging applications including teleoperation systems, surgical robots, prosthetics, VR/AR, games, and industry. For haptic feedback, the invented haptic glove can reproduce the force interaction between an industrial robotic gripper/prosthesis and grasped objects felt at a remote location by users. The robotic gripper/prosthesis will have integrated 3D force sensors and can provide interaction forces that then utilize as control input to the SSDs and amplified by a gain factor. In addition, the invented glove can be used in other teleoperation applications and surgical robots, 3D force interaction sensed from force sensors in surgical tools and grippers can be visualized on a surgeon's fingertips via multiple SSDs as shown in FIG. 40. This technology can overcome the lack of haptic feedback in existing surgical robotic systems. With the aid of SSDs, surgeons are able to adjust how much force applied to organs to make sure there is no damage. This technology is also needed in needle insertion for soft tissue and medical palpation in robotic surgery. In other embodiments, the wearable haptic glove with integrated single or multiple 3-axis SSDs can be used to provide haptic sensation for medical examination of the human organs such as inner walls of blood vessels, hearts, anus, colons, stomach, ears, bladders, and others with real-time force feedbacks from force sensors integrated at the tip of surgical robotic arms.

v. Telehealth Medical Services

In response to COVID-19 and other infectious diseases, telehealth is shown as a key method to limit face-to-face contact of patients and health professionals in hospital. It will also help vulnerable doctors to continue to deliver medical services to their patients. In an embodiment, SSDs are attached to the tele-controller system that controls a robotic arm for doing an ultrasound inspection on a patient as shown in FIG. 41. The SSDs will provide cutaneous haptic feedback to the surgeon's hands, which allows them to apply exact force on the patient's abdominal wall and see clear results as doing directly.

C. Touchable Haptic Applications

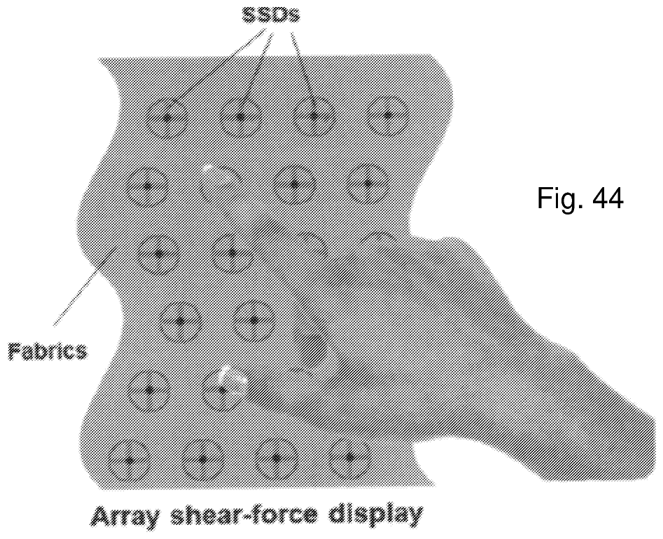

The array of miniature SSDs can be used a touchable shear-force display, which is purely cutaneous device that distributes various shear force in entire surface. The invented interfaces can be used to reproduce the interaction force between user and virtual objects in gaming or convey new type of textual language that is similar to braille alphabets but in term of a tactile book. FIG. 44 shows an array shear-force display

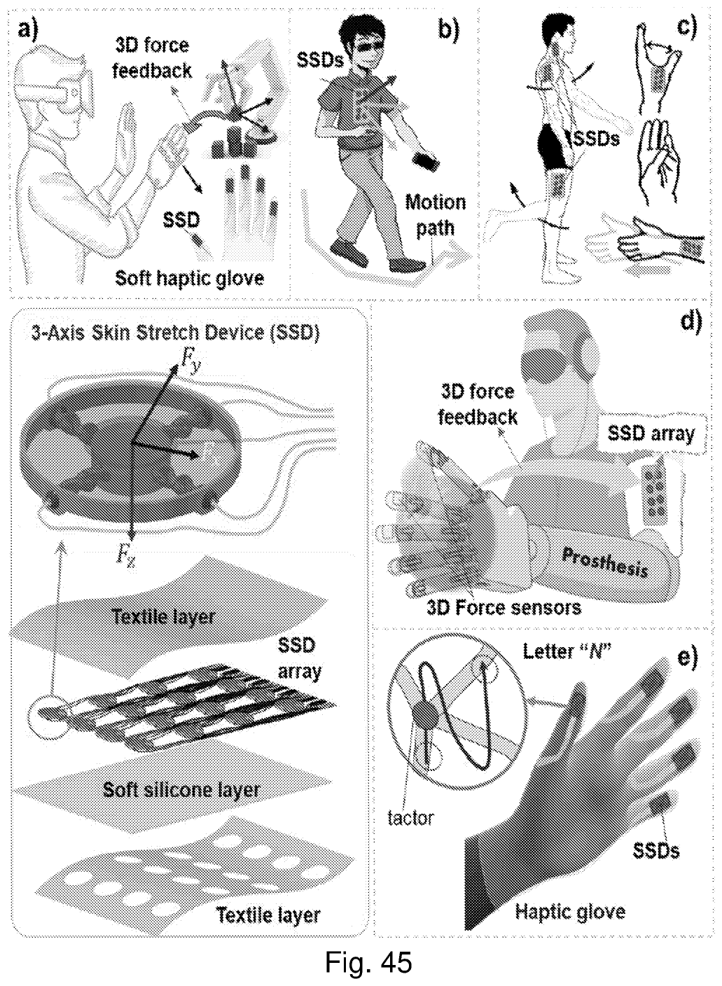

FIG. 45 shows potential application areas for soft skin stretch devices; a) haptic feedback for teleoperation system, b) navigational assistance for the visually impaired or older people, c) haptic motion guidance for human limbs and body, d) force feedback display for prosthesis

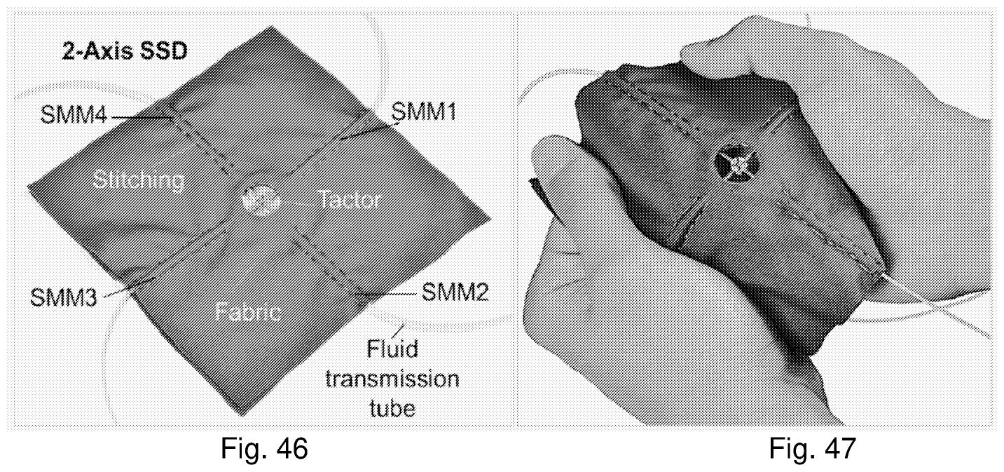

FIG. 46 and FIG. 47 show a skin stretch device driven by four soft microtubule muscles; Four SMMs-driven 2-axis SSDs with an optimal iPSE value of 100% for the muscles is fabricated (FIG. 11) where four DC micromotors (model 3272G024CR with encoder 1E3-1024, Faulhaber, Germany) connected to ball screw mechanisms (MISUMI, Japan) are used to control the SMMs via BD Luer-Lok™ 1-mL syringes as the preferred embodiment. Fluid pressure is monitored using a pressure sensor (40PC Series Sensor, Honeywell, USA). Experiments are conducted with a maximum displacement of the 2-axis SSD tactor within its working area via the evaluation of its reachability for eight different directions.

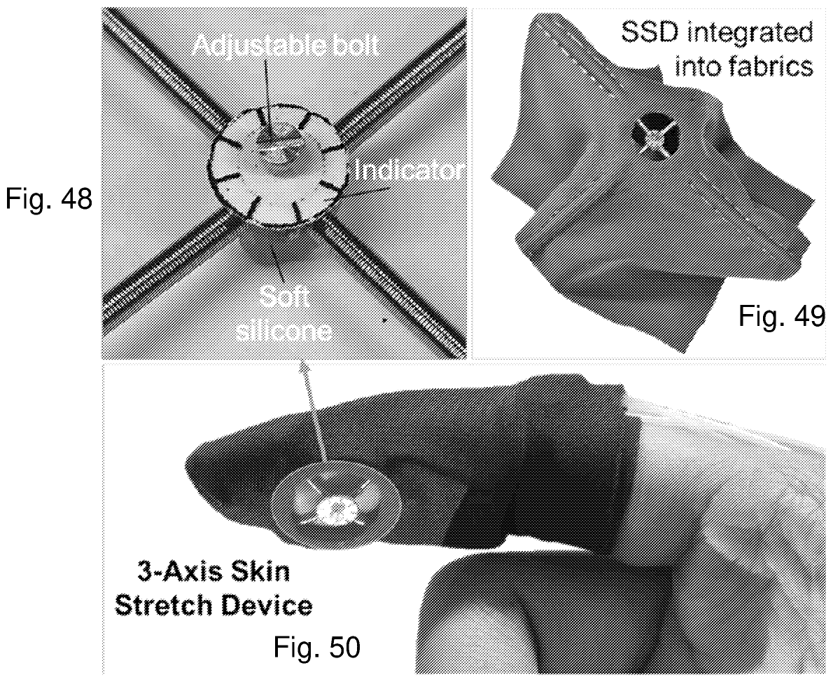

FIG. 48 to FIG. 50 shows a further embodiment of a soft tactor integrated into a 3-axis SSD in flexible fabrics.

In the SSD, tactor plays a vital part to connect the four SMMs and to induce stretch sensation to the skin. To deliver accurate haptic stimuli to the skin, it is essential to control the mechanical contact state which is especially challenging for current SSDs. Adhesion of the tactor with the skin will thus be achieved via a soft silicone surface. In order to control the normal force Fz in the z-axis, an adjustable mechanism is used. The soft tactor consists of a soft silicone layer at the bottom connected to the tactor head via a 3D printed bolt. The adjustable tactor can control the strength of contact friction forces, enabling a wide range of haptic stimuli. With this new design, the SSD is able to impart a directional shear force that can displace, deform, and stretch the skin, depending on the application such as textual communication, directional cues for motion guidance, and 3D force feedback display. To connect one end of the four SMMs to the tactor base, an adhesive glue (LOCTITE®, USA) is utilized while the other end is connected to a commercial fluid microtube (Cole-Parmer, USA). The 3D printed base is designed in a way that aligns the four force vectors from the SMM in the x-y plane. The soft tactor head which is made of ReoFlex 30 urethane rubber (Smooth-On, Inc., USA) provides high friction to improve the effect of skin stretch. The adjustable mechanism is a 3D printed bolt that can travel along the z-axis via a threaded hole in the tactor base. To achieve the desired contact force, users just simply adjust the bolt via its head. This mechanism also possesses an indicator that has a linear relation with the contact force via an adjustable angle of the bolt.

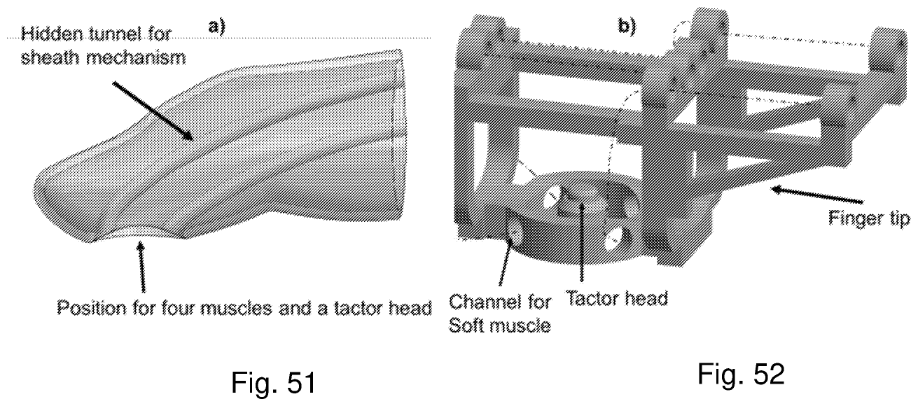

FIG. 51 shows further embodiments of a housing for a SSD including a tactor using a soft silicon housing. Fig. shows using a 3D-printed rigid frame housing.

A. Design and Fabrication of Soft Microtubule Muscle

Existing actuators such as electromagnetic actuators, hydraulic pistons, tendon-sheath mechanisms, piezoelectric actuators are inappropriate for hand-worn SSDs due to the occlusion by rigid components that are not able to match with the geometric complexity of the skin surface. The present invention relates to a new type of SMMs that can transmit force and motion from a far distance without scarifying its input energy. The new SMMs are driven by fluid pressure source that consists of a flexible silicone microtube or microtubule and a hollow constrained layer. The obtained muscle composite is lengthened or shortened under the applied fluid pressure. A fluid transmission tube or guide tube which connects the inner channel of the soft microtubule to a miniature syringe and a micro DC motor via a linear ball screw system is used to supply the pressure. By constraining the radial expansion of the flexible microtubule via a constrained layer, it is possible to mechanically program the muscle to perform desired motions and forces.

In one embodiment, the flexible silicone microtubule can be fabricated from a rolling coating process. In another embodiment, the microtube can be manufactured by dip-coating techniques or tube extrusion method with silicone materials ranging from Ecoflex, PDMS, Exosil, Reoflex, Vytaflex, Latex rubber. The microtube can be off-the-shelf commercial medical microtube or any stretchable ones. The microtube is inserted into a constrained layer (a hollow micro-coil made from inextensible fibers or wrinkled hollow fabrics/thin tubes that can be only elongated along with its axial directions). In one embodiment, the micro-coils a type of stainless steel that can store elastic energy under the applied strain. In another embodiment, the coil can be fabricated from the fishing line, wrapped around a micro carbon fiber, following by heating in an oven to form the final coil shape. The constrained coil (inextensible fiber) can be used as a miniature extension spring like ones from MCMaster-Carr, USA, which provides a consistent diameter and axial force along the fiber length. The constrained wrinkled outer layer can be fabricated by wrinkling a hollow inextensible tube such as ultrathin-wall PET, Nylon tubes or any inextensible fabrics. In an embodiment, the outer micro-coil of the muscle can be sewing threads or fishing lines that are gently wrapped around the flexible silicone tube circumference with a rigid rode inside to prevent collapse in the silicone tube wall. To form the coil, the constrained layer is produced by wrapping the fishing line around a carbon fiber rode in a helical shape, following by heating in an oven set at 130 degrees Celsius in two hours.

Once the soft microtube is completely inserted into the constrained outer layer, its one end is tied a knot and permanently adhered into the one end of constrained layer by an adhesive glue (LOCTITE®, USA or any other adhesive glues) while the other end is connected to a commercial fluid tube (Cole-Parmer, USA or any non-stretchable and flexible microtube such as PET tubes). The fluid transmission tube is then connected to a miniature syringe via a blunt needle. To remove the air bubbles that are trapped inside the soft microtube and fluid transmission tube, the whole composites are degassed in a vacuum chamber for 30 minutes (Binder—VD115, Binder, USA) until the air bubbles are completely disappeared. When fluid pressure is applied to the actuation channel, the SMM will be lengthened from position A at length L₀ to B at length L with a displacement x which is due to the circumferential constraint by inextensible fibers around the channels. At point B, it stores elastic energy. If a load is connected to the end of the actuator at position B and the fluid is removed, the stored elastic energy is released, allowing the actuator to apply a force against the load and bring it back to the position A. The higher pressure is applied, the higher elastic energy is stored and then a higher contraction force is achieved (at position C with length Lₘₐₓ and displacement xₘₐₓ).

In one prototype, the flexible silicone tube (microtubule) has an outer diameter of 1.1 mm, an inner diameter of 0.7 mm, and a working length of 50 mm. The invented SMM is scalable and therefore its diameters and length can be varied to adapt to specific applications. The corresponding constrained coil has an outer diameter of 1.5 mm, an inner diameter of 1.1 mm that covers the entire length of the silicone microtube. In this embodiment, the use of stainless-steel coil offers many advantages including high durability and high forces compared to others such as fishing line, sewing thread or nylon coils.

In another embodiment, the actuation muscles for the devices can be obtained by twisting at least two SMMs. This embodiment will enhance the generated force compared to a single configuration.

Analytical Model for the SMM

The soft microtubule muscles are modelled with the assumption that the water pressure is uniformly applied to the inner flexible rubber tube and there are no radical expansions during the operation. A Poisson's ratio of 0.5 is used for the soft silicone tube in the sense that the volume of the silicone tube is constant during its working period. Therefore, the cross-sectional area of the silicone tube is reduced if it is elongated under the applied fluid pressure and this relation can be expressed by:

π(dₒ²−dᵢ²)(1+ϵₗ)=π(dₒ²−dᵢ₀²), (3)

where d, dᵢ, are the outer diameter, inner diameter during the working period, and dᵢ₀, d₀ are the inner diameter and outer diameter at the initial state of the silicone tube, respectively. ϵₗ is the strain in the axial direction.

In the axial direction, the total elastic force in the coil F_{c} and the tubing Fᵣ is balanced by the external force, Fₑₓₜ, dissipative forces, F_{dis}, include viscosity and friction and the force due to the pressure inside the muscle, Fₚ:

Fₑₓₜ=F_{c}+Fᵣ−Fₚ+F_{dis} (4)

In embodiments using wrinkled hollow fabrics or sewing threads as the constrained layer, F_{c} is approximate to zero because the stiffness of the constrained layer is zero in the axial direction. In this case, the elastic force will only generate from flexible silicone microtubule.

The elastic force in the coil is written in Eq. 3, where x denotes the elongation of the actuator and k_{c} is the spring coefficient of the constrained coil.

F_{c}=k_{c}x (5)

The elastic force in the rubber tubing can be expressed by:

$${F}_{r} = {EA}_{d 0} ( 1 - \frac{1}{1 + x / {L}_{0}} ) \qquad ( 6 )$$

where, A_{d}=0.25π(d²−dᵢ²), A_{d0}=0.25π(dₒ²−dᵢ₀²), E is Young's modulus, and L₀ is the nominal length of the muscle.

The driving force Fₚ can be estimated as:

Fₚ=A_{di}P=0.25πdᵢ² (7)

where P is the fluidic pressure inside the rubber tube.

The dissipative forces, F_{dis}, include hydrodynamic flow resistance, F_{dis,hyd}, and dry friction at the tube fabric interface, F_{dis,dry} as shown:

F_{dis}=F_{dis,hyd}+F_{dis,dry} (8)

However, F_{dis,hyd} and F_{dis,dry} can be neglected under the assumption that the relative motion of the tube and the coil is small and the actuator is working in quasi-hydrostatic operation. Therefore, the net external force exerted by the actuator is:

$${F}_{ext} = {F}_{c} + {F}_{r} - {F}_{p} = {k}_{c} x + {EA}_{d 0} ( 1 - \frac{1}{1 + \frac{x}{{L}_{0}}} ) - {A}_{di} P \qquad ( 9 )$$

The external force Fₑₓₜ, achieves its maximum value at a maximum elongation xₘₐₓ when the fluid pressure P=0. This maximum value is extremely important in this skin-stretch design, which determines how much force is applied to the tactor. At a fluid pressure, P, the muscle elongates to a distance, x, and Fₑₓₜ=0, the relationship between the elongation distance and the fluid pressure is given:

$$P = \frac{{k}_{c} {x}^{2} + {k}_{c} {L}_{0} x + {EA}_{d 0} x}{{A}_{d 0} {L}_{0}} \qquad ( 10 )$$

Assume that all mechanical energy from the syringe plunger, Wₚ is transferred to the elastic energy of the coil, W_{c}, and the silicone tube, Wᵣ, and the energy lost, Wₗ, due to sliding friction through the working fluid. Mechanical energy from the syringe plunger is equal to work generated in the fluid system. The energy relation can be given by:

$${W}_{p} = {W}_{c} + {W}_{r} + {W}_{l} \qquad ( 11 )$$

$$PV = \frac{1}{2} {k}_{c} {x}^{2} + \frac{1}{2} {k}_{r} {x}^{2} + {W}_{l} \qquad \text{(12)}$$

where V is the volume of fluid that is pushed into the actuator, and kᵣ is the instantaneous spring constant of the rubber tubing with the equation as shown:

$${k}_{r} = \frac{{EA}_{r 0} {L}_{0}}{{( {L}_{0} + x )}^{2}} \qquad ( 13 )$$

Finally, there is a relationship between the liquid volume and the elongation of the actuator.

$$V = \frac{\frac{1}{2} {k}_{c} {x}^{2} + \frac{1}{2} {k}_{r} {x}^{2} + {W}_{l}}{P} \qquad ( 14 )$$

In the later sections, the actual results and the predictions of the above analytical model are compared with the geometric dimensions of each component and mechanical parameters from practical measurements such as k_{c}=9.902 N/m, E=1.3 MPa, Aᵣ₀=2.26×10−6 m2, l₀=45 mm for the preferred embodiment with the stainless-steel coil and microtubule. Similarly, the comparison can be conducted for other embodiments.

### Interpretation

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

As used herein, the term “exemplary” is used in the sense of providing examples, as opposed to indicating quality. That is, an “exemplary embodiment” is an embodiment provided as an example, as opposed to necessarily being an embodiment of exemplary quality.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

1. An actuator device comprising:
a central housing frame having a planar cavity;
a central tactor surface located within said planar cavity;
a series of controllable synthetic muscles elements attached to the housing frame and to the central tactor surface and activated to allow for controlled movement of around the planar cavity; and
activation means for activating the controllable synthetic muscles so as to move the tactor around the cavity in a controlled manner.

2. The actuator as claimed in claim 1, wherein said synthetic muscles include at least one microtubular actuator which comprises a central elongated flexible conduit surrounded by a helical coil and the synthetic muscles are activated by controlling the pressure of a fluid within the conduit.

3. The actuator as claimed in claim 1, wherein said synthetic muscles include multiple microtubular actuators intertwined or twisted together.

4. (canceled)

5. The actuator as claimed in claim 1, wherein said central housing frame is attached to a flexible planar sheet material.

6-7. (canceled)

8. The actuator as claimed in claim 1, further comprising an out of axis resiliently deformable element providing out of axis movement of the tactor upon activation of the synthetic muscle elements.

9. (canceled)

10. The actuator as claimed in claim 8, wherein said out of axis resiliently deformable element is independently controlled to provide out of axis deformation.

11. The actuator as claimed in claim 1, wherein the number of synthetic muscle elements is at least four, interconnecting the central tactor surface with the frame.

12. The actuator as claimed in claim 1, wherein said synthetic muscle elements are formed within a flexible sheet.

13. An array of actuators as claimed in claim 1 interposed between an inner and outer flexible sheet.

14. A haptic device comprising:
an array of soft hydraulic skin stretch devices,
soft microtubule muscles configured to control the skin stretch devices.

15. The haptic device as defined in claim 14, further comprising a tactor configured to deliver 3-axis tangential forces from the soft microtubule muscles to the skin.

16-18. (canceled)

19. The haptic device as defined in claim 1, wherein in use the skin stretch devices are able to deliver forces that are both normal and tangential to the skin.

20. A soft microtubule muscle driven by a fluid pressure source that consists of a flexible silicone microtube or microtubule and a hollow micro-coil which is made from inextensible fibers.

21. The actuator as claimed in claim 2, wherein the number of synthetic muscle elements is at least four, interconnecting the central tactor surface with the frame.

22. The actuator as claimed in claim 2, wherein said synthetic muscle elements are formed within a flexible sheet.

23. An array of actuators as claimed in claim 2 interposed between an inner and outer flexible sheet.
